(19) United States
(12) Patent Application Publication
(10) Pub. No.: US 2011/0143344 A1
(43) Pub. Date: Jun. 16, 2011

(54) GENETIC POLYMORPHISMS AND SUBSTANCE DEPENDENCE

(75) Inventors: Alison M. Goate, St. Louis, MO (US); Laura J. Bierut, St. Louis, MO (US); Jen-Chyong C. Wang, St. Louis, MO (US)

(73) Assignee: THE WASHINGTON UNIVERSITY, St. Louis, MO (US)

(21) Appl. No.: 12/955,306

(22) Filed: Nov. 29, 2010

Related U.S. Application Data
(63) Continuation-in-part of application No. 12/246,399, filed on Oct. 6, 2008, now abandoned.
Application No. 12/246,399 is a continuation-in-part of application No. 11/681,177, filed on Mar. 1, 2007.
(60) Provisional application No. 60/978,030, filed on Oct. 5, 2007.
(60) Provisional application No. 60/778,597, filed on Mar. 1, 2006.
(60) Provisional application No. 60/811,318, filed on Jun. 6, 2006.

(51) Int. Cl. C12Q 1/68; C07H 21/00

(52) U.S. Cl. 435/6.11; 435/6.13; 536/23.1

(57) ABSTRACT

The invention encompasses methods for identifying subjects at risk for substance dependence by detecting the presence of polymorphism in the CHRNA5-CHRNA3-CHRNB4 gene cluster and the CHRNA4 gene. The invention also encompasses determining the response of a subject to a therapeutic substance, treating substance dependence in a subject, and evaluating the response of a subject to a substance cessation treatment.

### CROSS REFERENCE TO RELATED APPLICATIONS

This application is a Continuation in Part of U.S. application Ser. No. 12/246,399, filed Oct. 6, 2008, which claims the priority of U.S. provisional application No. 60/978,030, filed Oct. 5, 2007, and this application is a Continuation in Part of U.S. application Ser. No. 11/681,177, which claims the priority of U.S. provisional application No. 60/778,597, filed Mar. 1, 2006 and the priority of U.S. provisional application No. 60/811,318, filed Jun. 6, 2006, each of which is hereby incorporated by reference in its entirety.

### GOVERNMENTAL RIGHTS

The present invention was supported by funding from the National Institutes of Health, NIAAA (2U10 AA08403) and NIDA (R01 DA19963). The United States Government has certain rights in this invention.

### FIELD OF THE INVENTION

The invention encompasses, in part, methods for identifying subjects at risk for substance dependence.

### BACKGROUND OF THE INVENTION

Dependence on alcohol, nicotine and other substances continues to be one of the most serious public health problems worldwide. The discovery and characterization of addiction susceptibility genes greatly improves the identification of individuals at high risk of substance addiction in order to target them for therapeutic trials and disease-modifying therapies.

### SUMMARY OF THE INVENTION

One aspect of the invention encompasses a method for identifying a subject at risk for substance dependence. The method comprises detecting, in a sample from the subject, the presence of at least one polymorphism in the CHRNA5-CHRNA3-CHRNB4 gene cluster and the CHRNA4 gene. The polymorphisms typically have a correlation value of 0.7 or greater with each other. Generally speaking, the presence of one of the alleles of the polymorphism is associated with increased risk for substance dependence.

Another aspect of the invention encompasses a method for determining the response of a subject to a therapeutic substance. The method comprises detecting, in a sample from the subject, the presence of at least one polymorphism in the CHRNA5-CHRNA3-CHRNB4 gene cluster and the CHRNA4 gene. Generally speaking, the presence of a first allele of the polymorphism is associated with a first response and the presence of a second allele of the polymorphism is associated with a second response.

Yet another aspect of the invention encompasses a method for treating substance dependence in a subject. The method comprises administering to the subject an agent that alters the level of the alpha 5 subunit of the nicotinic acetylcholine receptor and/or alters the activity of the alpha 5 subunit of the nicotinic acetylcholine receptor.

Still another aspect of the invention encompasses a method for evaluating the response of a subject to a substance cessation treatment. The method comprises determining the level of CHRNA5 messenger RNA in a first and a second sample from the subject. The first sample is collected before the start of the substance cessation treatment, and the second sample is collected during or after the substance cessation treatment. A decreased level of CHRNA5 messenger RNA in the second sample relative to the first sample indicates the subject is responding to the substance cessation treatment.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

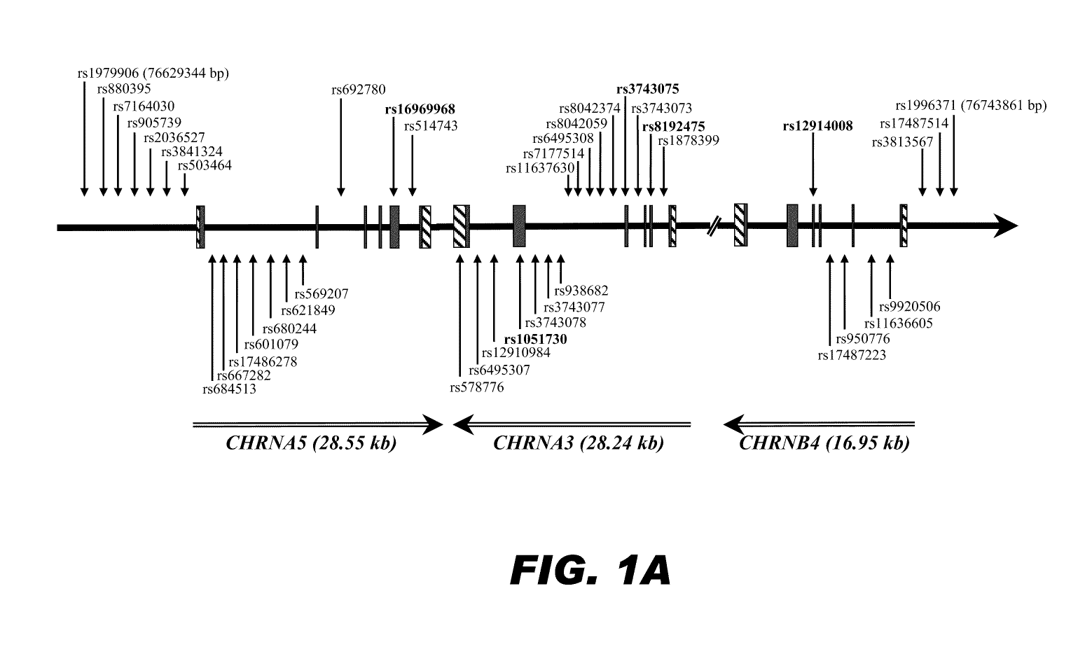

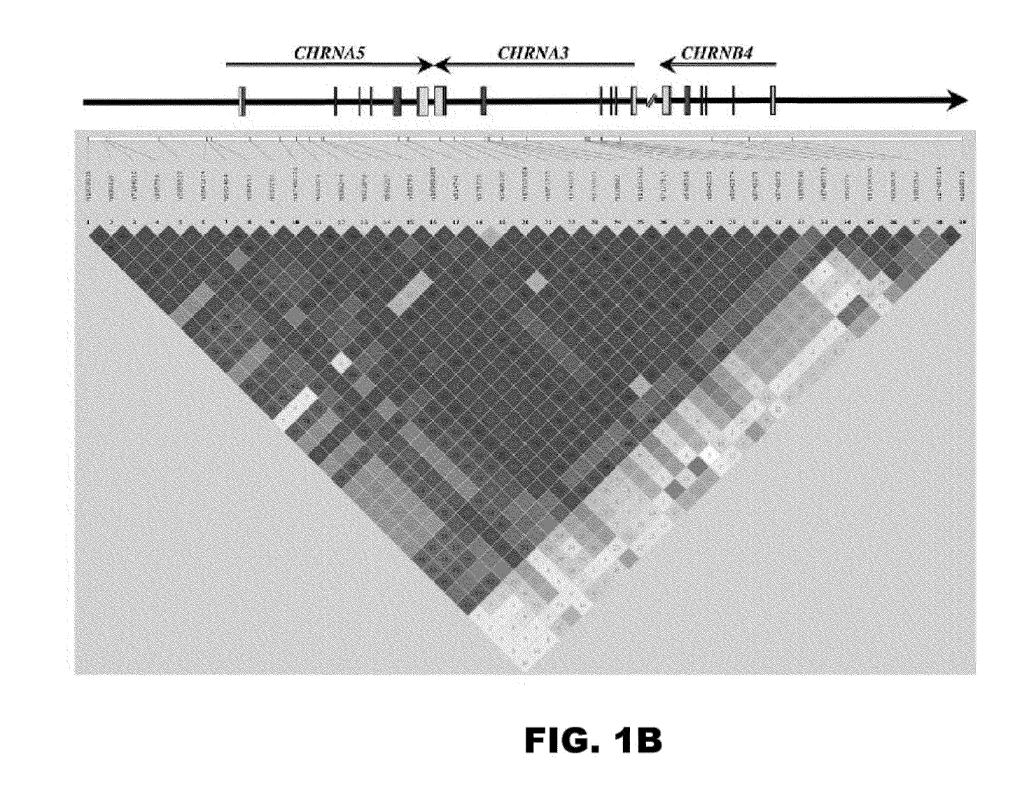

FIG. 1A diagrams the location of genotyped SNPs across the cluster of CHRNA5-CHRNA3-CHRNB4 genes. The dark gray boxes represent coding exons. The light gray boxes represent 5′ and 3′ UTRs. Diagram is not drawn to scale.

FIG. 1B presents the pair-wise linkage disequilibrium among single nucleotide polymorphisms (SNPs) in the region of CHRNA5-CHRNA3-CHRNB4 genes in European Americans from the Collaboration of the Genetics of Alcoholism (COGA) dataset.

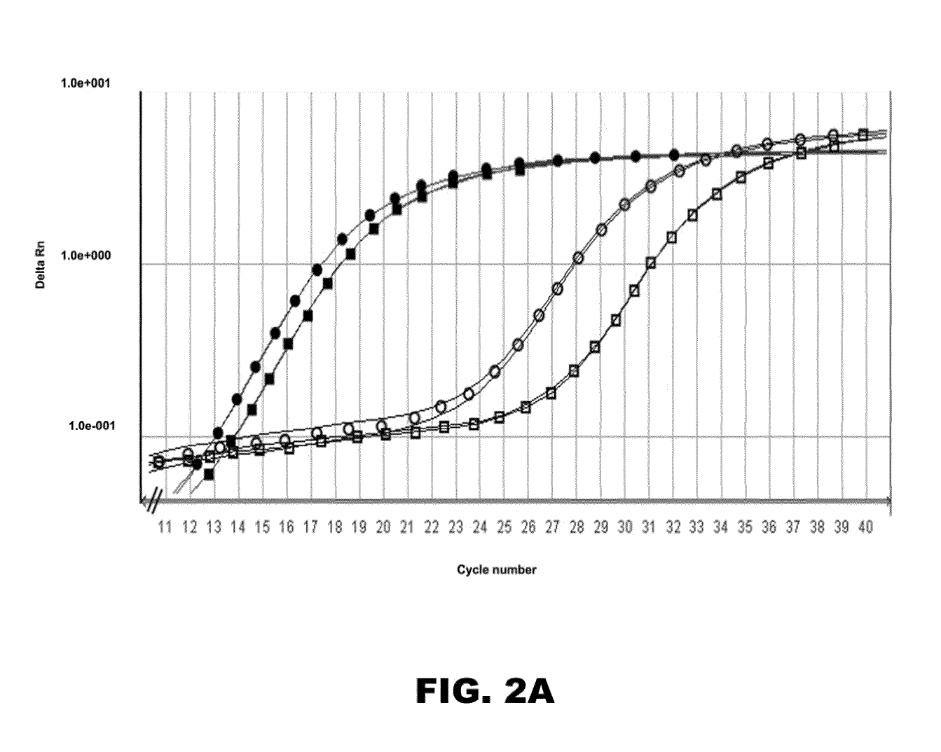

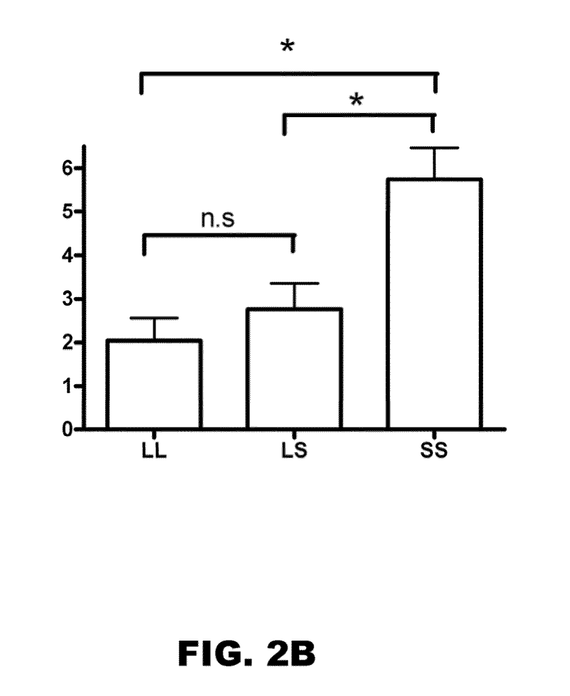

FIG. 2 illustrates the effect of the 22 by insertion/deletion promoter polymorphism (SNP 6/rs3841324), or a variant in high positive linkage disequilibrium with this polymorphism on CHRNA5 gene expression. (A) The ARn vs. cycle plot showing relative total expression of CHRNA5 (open circles and open squares) and GAPDH (solid circles and solid squares). Circles represent samples homozygous for deletion and squares represent samples homozygous for insertion. The level of expression was calculated from the Ct value (the cycle at which the fluorescence intensity rises above a threshold) and was normalized by taking GAPDH as a reference. (B) Mann-Whitney U statistic, 2-tailed analysis of CHRNA5 total expression in subjects with homozygous insertion (LL), subjects with homozygous deletion (SS), and heterozygous subjects (LS). Y-axis represents the relative expression level taking an arbitrary reference sample as 1. Mean±standard deviation is shown; * indicates p value <0.05.

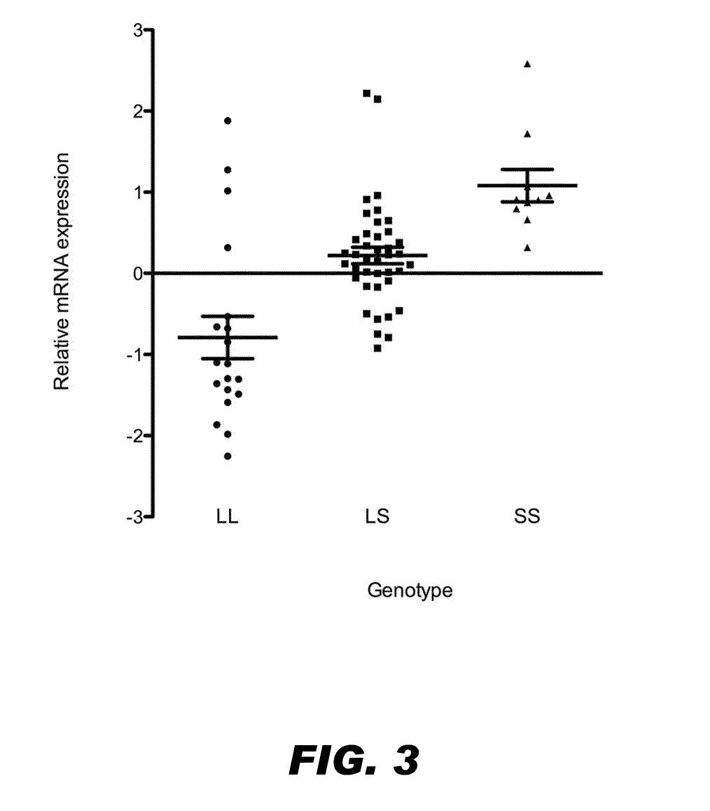

FIG. 3 illustrates the effect of the 22 bp insertion/deletion promoter polymorphism (SNP6/rs3841324), or a variant in high positive linkage disequilibrium with this polymorphism on CHRNA5 gene expression. The graph depicts relative CHRNA5 mRNA expression in subjects with homozygous insertion (LL), subjects with homozygous deletion (SS), and heterozygous subjects (LS). The y-axis represents the relative mRNA expression corrected for covariance (residuals) of gender and site.

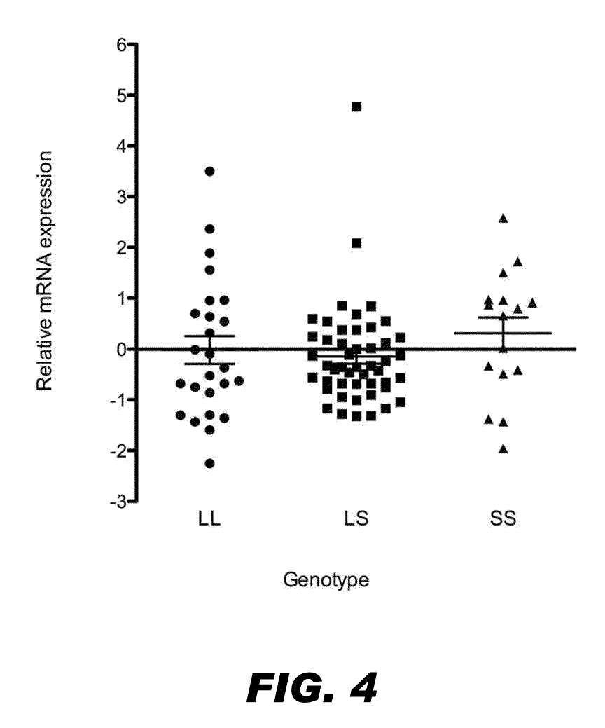

FIG. 4 illustrates the effect of the 22 bp insertion/deletion promoter polymorphism (SNP6/rs3841324), or a variant in high positive linkage disequilibrium with this polymorphism on CHRNA3 gene expression. The graph depicts relative CHRNA3 mRNA expression in subjects with homozygous insertion (LL), subjects with homozygous deletion (SS), and heterozygous subjects (LS). The y-axis represents the relative mRNA expression corrected for covariance (residuals) of gender and site.

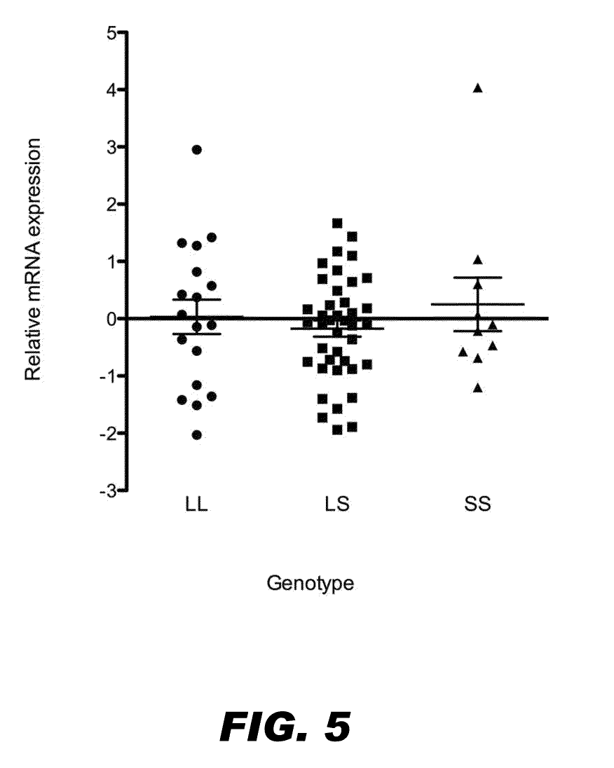

FIG. 5 illustrates the effect of the 22 bp insertion/deletion promoter polymorphism (SNP6/rs3841324), or a variant in high positive linkage disequilibrium with this polymorphism on CHRNB4 gene expression. The graph depicts relative CHRNB4 mRNA expression in subjects with homozygous insertion (LL), subjects with homozygous deletion (SS), and heterozygous subjects (LS). The y-axis represents the relative mRNA expression corrected for covariance (residuals) of gender and site.

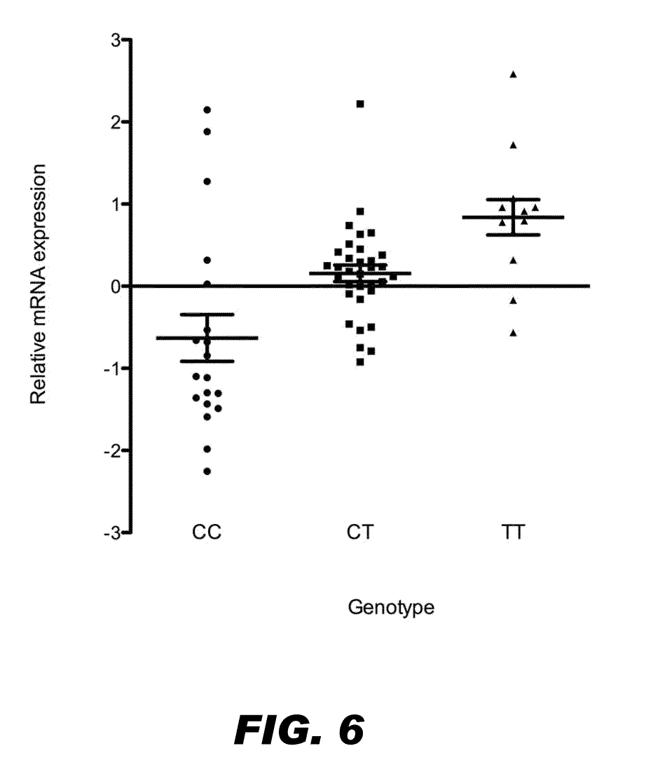

FIG. 6 illustrates the effect of the rs588765 polymorphism, or a variant in high positive linkage disequilibrium with this polymorphism on CHRNA5 gene expression. The graph depicts relative CHRNA5 mRNA expression in subjects with the homozygous C variant, subjects with the homozygous T variant, and heterozygous subjects. The y-axis represents the relative mRNA expression corrected for covariance (residuals) of gender and site.

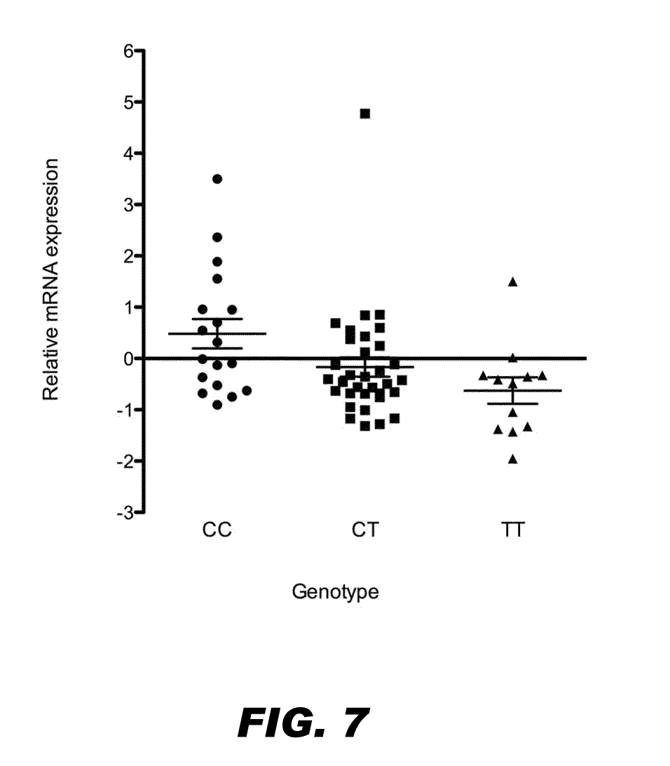

FIG. 7 illustrates the effect of the rs588765 polymorphism, or a variant in high positive linkage disequilibrium with this polymorphism on CHRNA3 gene expression. The graph depicts relative CHRNA3 mRNA expression in subjects with the homozygous C variant, subjects with the homozygous T variant, and heterozygous subjects. The y-axis represents the relative mRNA expression corrected for covariance (residuals) of gender and site.

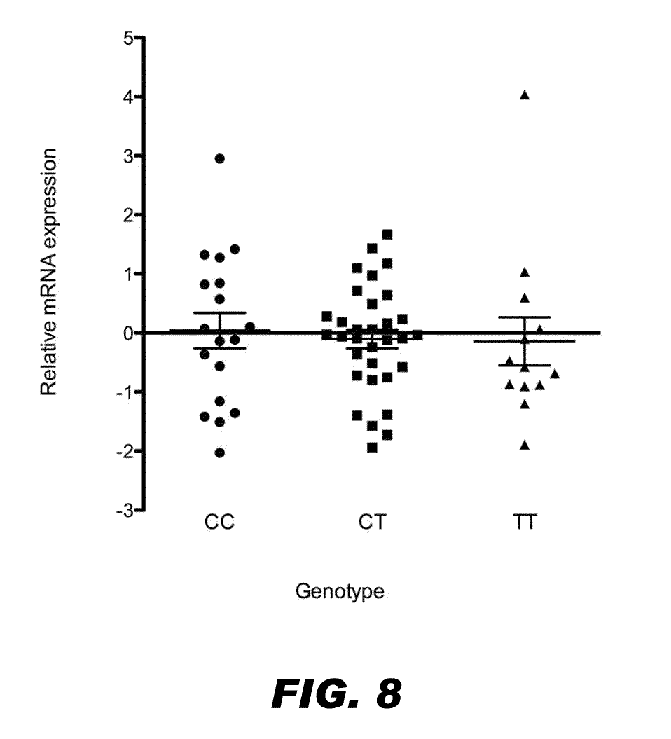

FIG. 8 illustrates the effect of the rs588765 polymorphism, or a variant in high positive linkage disequilibrium with this polymorphism on CHRNB4 gene expression. The graph depicts relative CHRNB4 mRNA expression in subjects with the homozygous C variant, subjects with the homozygous T variant, and heterozygous subjects. The y-axis represents the relative mRNA expression corrected for covariance (residuals) of gender and site.

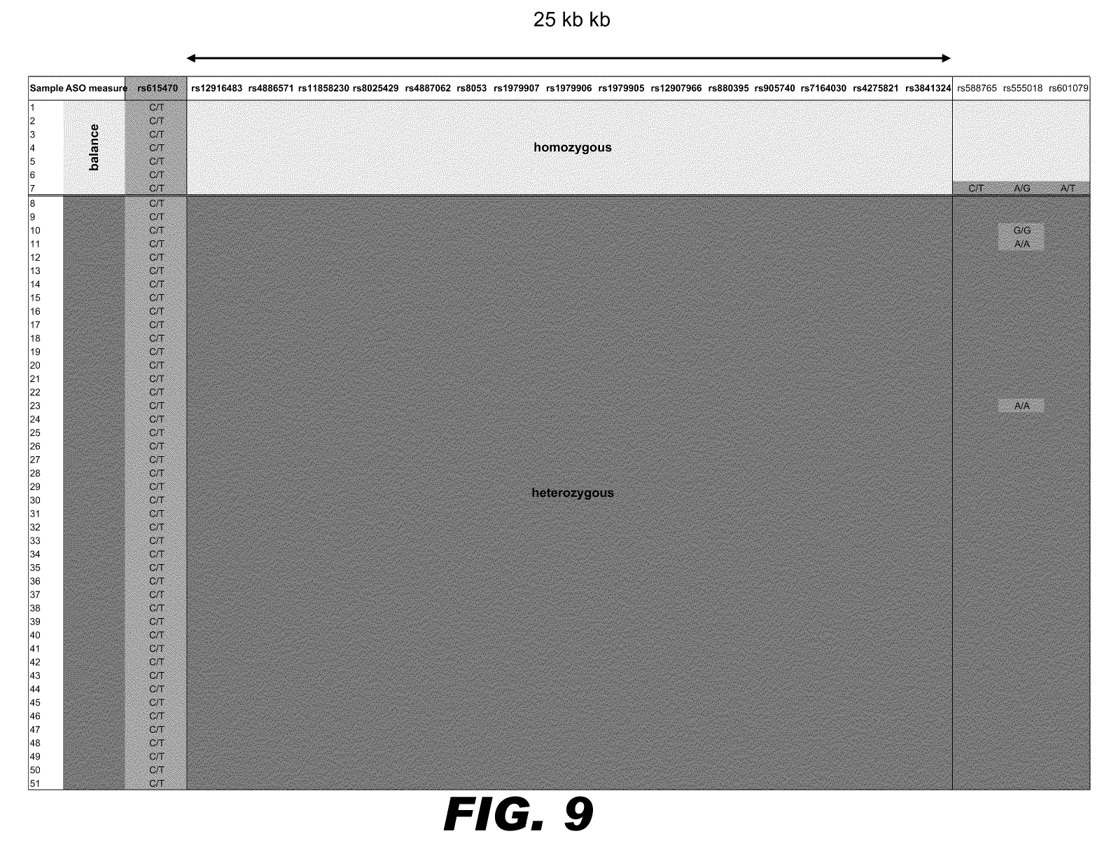

FIG. 9 depicts allele-specific expression of CHRNA5 mRNA in brain tissues from European Americans and Australians.

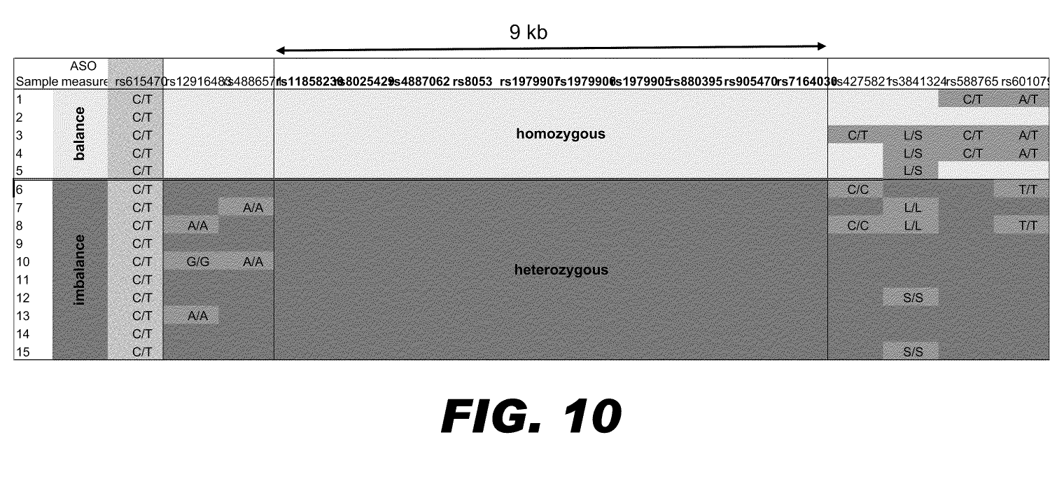

FIG. 10 depicts allele-specific expression of CHRNA5 mRNA in brain tissues from African Americans.

### DETAILED DESCRIPTION OF THE INVENTION

### (I) Method for Identifying a Subject at Risk for Substance Dependence

One aspect of the present invention provides a method for identifying a subject at risk for substance dependence. The method comprises detecting in a sample from the subject the presence of at least one polymorphism in the CHRNA5-CHRNA3-CHRNB4 gene cluster and the CHRNA4 gene, wherein the polymorphisms have a correlation value (R²) of 0.7 or greater with each other. The presence of one of the alleles of the polymorphism is associated with increased risk for substance dependence.

In one embodiment, the polymorphism may be selected from the group consisting SNP 1, SNP 2, SNP 3, SNP 4, SNP5, SNP 6, SNP 11, SNP 12, SNP 15, SNP 17, SNP 19, SNP 23, SNP 30, SNP 31, SNP 32. SNP 40, SNP, 41, SNP 42, SNP 43, SNP 44, SNP 45, SNP 46 and a combination thereof. These SNPs are identified in Table A.

TABLE A
Polymorphisms Associated with Substance Dependence.

| SNP | Gene | dbSNP reference |
|---|---|---|
| 1 | Upstream of CHRNA5 | rs1979906 |
| 2 | Upstream of CHRNA5 | rs880395 |
| 3 | Upstream of CHRNA5 | rs7164030 |
| 4 | CHRNA5 | rs16969968 |
| 5 | CHRNA5 | rs514743 |
| 6 | CHRNA5 | rs3841324 |
| 11 | CHRNA5 | rs601079 |
| 12 | CHRNA5 | rs680244 |
| 15 | CHRNA5 | rs692780 |
| 17 | CHRNA5 | rs514743 |
| 19 | CHRNA3 | rs6495307 |
| 23 | CHRNA3 | rs3743077 |
| 30 | CHRNA3 | rs3743075 |
| 31 | CHRNA3 | rs3743073 |
| 32 | CHRNA3 | rs1878399 |
| 40 | CHRNA3 | rs8192475 |
| 41 | CHRNB4 | rs12914008 |
| 42 | CHRNA4 | rs755203 |
| 43 | Upstream of PSMA4 | rs12916483 |
| 44 | PSMA4 | rs4886571 |
| 45 | Upstream of CHRNA5 | rs8053 |
| 46 | Upstream of CHRNA5 | rs12907966 |

In another embodiment, the polymorphism may be selected from the group consisting of a) SNP 6, wherein the absence of a 22 base pair insertion is associated with increased risk for substance dependence, b) SNP 11, wherein the presence of A rather than T is associated with increased risk for substance dependence, c) SNP 12, wherein the presence of A rather than G is associated with increased risk for substance dependence, d) SNP 15, wherein the presence of G rather than C is associated with increased risk for substance dependence, e) SNP 19, wherein the presence of T rather than C is associated with increased risk for substance dependence, f) SNP 40, wherein the presence of G rather than A is associated with increased risk for substance dependence, g) SNP 41, wherein the presence of G rather than A is associated with increased risk for substance dependence, h) SNP 42, wherein the presence of T is associated with increased risk for substance dependence, i) SNP 43, wherein the presence of A rather than G is associated with increased risk for substance dependence, j) SNP 44, wherein the presence of A rather than G is associated with increased risk for substance dependence, k) SNP 45, wherein the presence of T rather than C is associated with increased risk for substance dependence, l) SNP 46, wherein the presence of T rather than C is associated with increased risk for substance dependence, or m) a combination thereof. In an exemplary embodiment, a SNP of the invention may be a SNP detailed in Table B below.

TABLE B
Polymorphisms Highly Associated with Substance Dependence

| SNP | dbSNP reference | Description |
|---|---|---|
| 6 | rs3841324 | 22 bp insertion/deletion in |
| | | promoter of CHRNA5 gene |
| 11 | rs601079 | intronic SNP in CHRNA5 gene |
| 12 | rs680244 | intronic SNP in CHRNA5 gene |
| 15 | rs692780 | intronic SNP in CHRNA5 gene |
| 19 | rs6495307 | intronic SNP in CHRNA3 gene |
| 40 | rs8192475 | exonic SNP in CHRNA3 gene |
| | | (nonsynonymous mutation) |
| 41 | rs12914008 | exonic SNP in CHRNB4 gene |
| | | (nonsynonymous mutation) |
| 42 | rs755203 | SNP in promoter region off |
| | | CHRNA4 gene |
| 43 | rs12916483 | upstream of PSMA4 |
| 44 | rs4886571 | intronic SNP in PSMA4 |
| 45 | rs8053 | upstream of CHRNA5 |
| 46 | rs12907966 | upstream of CHRNA5 |

In one embodiment, one polymorphism may be detected. In another embodiment, a combination of two polymorphisms may be detected. In yet another embodiment, a combination of three polymorphisms may be detected. In still another embodiment, a combination of four polymorphisms may be detected. In an alternate embodiment, a combination of five polymorphisms may be detected. In another alternate embodiment, a combination of six polymorphisms may be detected. In yet another embodiment, a combination of seven polymorphisms may be detected. In another alternate embodiment, a combination of seven or more polymorphisms may be detected.

In a preferred embodiment, SNP 2, SNP 3, SNP 6, SNP 43, SNP 44, SNP 45, or SNP 46 may be detected. In other preferred embodiments, a combination of SNP 2, SNP 3, SNP 6, SNP 43, SNP 44, SNP 45, or SNP 46 and at least one of the other polymorphisms may be detected. In still another preferred embodiment, SNP 4 and at least one of the other polymorphisms may be detected. Tables C and D list non-limiting examples of possible combinations.

TABLE C
Possible combinations

SNP 6, SNP 11
SNP 6, SNP 12
SNP 6, SNP 15
SNP 6, SNP 19
SNP 6, SNP 40
SNP 6, SNP 41
SNP 6, SNP 42
SNP 6, SNP 11, SNP 12
SNP 6, SNP 11, SNP 15
SNP 6, SNP 11, SNP 19
SNP 6, SNP 11, SNP 40
SNP 6, SNP 11, SNP 41
SNP 6, SNP 12, SNP 15
SNP 6, SNP 12, SNP 19
SNP 6, SNP 12, SNP 40
SNP 6, SNP 12, SNP 41
SNP 6, SNP 15, SNP 19
SNP 6, SNP 15, SNP 40
SNP 6, SNP 15, SNP 41
SNP 6, SNP 19, SNP 40
SNP 6, SNP 19, SNP 41
SNP 6, SNP 40, SNP 41
SNP 6, SNP 11, SNP 12, SNP 15
SNP 6, SNP 11, SNP 12, SNP 19
SNP 6, SNP 11, SNP 12, SNP 40
SNP 6, SNP 11, SNP 12, SNP 41
SNP 6, SNP 11, SNP 15, SNP 19
SNP 6, SNP 11, SNP 15, SNP 40
SNP 6, SNP 11, SNP 15, SNP 41
SNP 6, SNP 11, SNP 19, SNP 40
SNP 6, SNP 11, SNP 19, SNP 41
SNP 6, SNP 11, SNP 40, SNP 41
SNP 6, SNP 12, SNP 15, SNP 19
SNP 6, SNP 12, SNP 15, SNP 40
SNP 6, SNP 12, SNP 15, SNP 41
SNP 6, SNP 12, SNP 19, SNP 40
SNP 6, SNP 12, SNP 19, SNP 41
SNP 6, SNP 12, SNP 40, SNP 41
SNP 6, SNP 15, SNP 19, SNP 40
SNP 6, SNP 15, SNP 19, SNP 41
SNP 6, SNP 15, SNP 40, SNP 41
SNP 6, SNP 19, SNP 40, SNP 41
SNP 6, SNP 11, SNP 12, SNP 15, SNP 19
SNP 6, SNP 11, SNP 12, SNP 15, SNP 40
SNP 6, SNP 11, SNP 12, SNP 15, SNP 41
SNP 6, SNP 11, SNP 12, SNP 19, SNP 40
SNP 6, SNP 11, SNP 12, SNP 19, SNP 41
SNP 6, SNP 11, SNP 12, SNP 40, SNP 41
SNP 6, SNP 11, SNP 15, SNP 19, SNP 40
SNP 6, SNP 11, SNP 15, SNP 19, SNP 41
SNP 6, SNP 11, SNP 19, SNP 40, SNP 41
SNP 6, SNP 12, SNP 15, SNP 19, SNP 40
SNP 6, SNP 12, SNP 15, SNP 19, SNP 41
SNP 6, SNP 15, SNP 19, SNP 40, SNP 41
SNP 6, SNP 11, SNP 12, SNP 15, SNP 19, SNP 40
SNP 6, SNP 11, SNP 12, SNP 15, SNP 19, SNP 41
SNP 6, SNP 11 SNP 12, SNP 15, SNP 19, SNP 40, SNP 41

TABLE D
Further possible combinations

| | | | | | | |
|---|---|---|---|---|---|---|
| SNP2 | SNP3 | | | | | |
| SNP2 | SNP6 | | | | | |
| SNP2 | SNP43 | | | | | |
| SNP2 | SNP44 | | | | | |
| SNP2 | SNP45 | | | | | |
| SNP2 | SNP46 | | | | | |
| SNP3 | SNP6 | | | | | |
| SNP3 | SNP43 | | | | | |
| SNP3 | SNP44 | | | | | |
| SNP3 | SNP45 | | | | | |
| SNP3 | SNP46 | | | | | |
| SNP6 | SNP43 | | | | | |
| SNP6 | SNP44 | | | | | |
| SNP6 | SNP45 | | | | | |
| SNP6 | SNP46 | | | | | |
| SNP43 | SNP44 | | | | | |
| SNP43 | SNP45 | | | | | |
| SNP43 | SNP46 | | | | | |
| SNP44 | SNP45 | | | | | |
| SNP44 | SNP46 | | | | | |
| SNP45 | SNP46 | | | | | |
| SNP2 | SNP3 | SNP6 | | | | |
| SNP2 | SNP3 | SNP43 | | | | |
| SNP2 | SNP3 | SNP44 | | | | |
| SNP2 | SNP3 | SNP45 | | | | |
| SNP2 | SNP3 | SNP46 | | | | |
| SNP2 | SNP6 | SNP43 | | | | |
| SNP2 | SNP6 | SNP44 | | | | |
| SNP2 | SNP6 | SNP45 | | | | |
| SNP2 | SNP6 | SNP46 | | | | |
| SNP2 | SNP43 | SNP44 | | | | |
| SNP2 | SNP43 | SNP45 | | | | |
| SNP2 | SNP43 | SNP46 | | | | |
| SNP2 | SNP44 | SNP45 | | | | |
| SNP2 | SNP44 | SNP46 | | | | |
| SNP2 | SNP45 | SNP46 | | | | |
| SNP3 | SNP6 | SNP43 | | | | |
| SNP3 | SNP6 | SNP44 | | | | |
| SNP3 | SNP6 | SNP45 | | | | |
| SNP3 | SNP6 | SNP46 | | | | |
| SNP3 | SNP43 | SNP44 | | | | |
| SNP3 | SNP43 | SNP45 | | | | |
| SNP3 | SNP43 | SNP46 | | | | |
| SNP3 | SNP44 | SNP45 | | | | |
| SNP3 | SNP44 | SNP46 | | | | |
| SNP3 | SNP45 | SNP46 | | | | |
| SNP6 | SNP43 | SNP44 | | | | |
| SNP6 | SNP43 | SNP45 | | | | |
| SNP6 | SNP43 | SNP46 | | | | |
| SNP6 | SNP44 | SNP45 | | | | |
| SNP6 | SNP44 | SNP46 | | | | |
| SNP6 | SNP45 | SNP46 | | | | |
| SNP43 | SNP44 | SNP45 | | | | |
| SNP43 | SNP44 | SNP46 | | | | |
| SNP43 | SNP45 | SNP46 | | | | |
| SNP44 | SNP45 | SNP46 | | | | |
| SNP2 | SNP3 | SNP6 | SNP43 | | | |
| SNP2 | SNP3 | SNP6 | SNP44 | | | |
| SNP2 | SNP3 | SNP6 | SNP45 | | | |
| SNP2 | SNP3 | SNP6 | SNP46 | | | |
| SNP2 | SNP3 | SNP43 | SNP44 | | | |
| SNP2 | SNP3 | SNP43 | SNP45 | | | |
| SNP2 | SNP3 | SNP43 | SNP46 | | | |
| SNP2 | SNP3 | SNP44 | SNP45 | | | |
| SNP2 | SNP3 | SNP44 | SNP46 | | | |
| SNP2 | SNP3 | SNP45 | SNP46 | | | |
| SNP2 | SNP6 | SNP43 | SNP44 | | | |
| SNP2 | SNP6 | SNP43 | SNP45 | | | |
| SNP2 | SNP6 | SNP43 | SNP46 | | | |
| SNP2 | SNP6 | SNP44 | SNP45 | | | |
| SNP2 | SNP6 | SNP44 | SNP46 | | | |
| SNP2 | SNP6 | SNP45 | SNP46 | | | |
| SNP2 | SNP43 | SNP44 | SNP45 | | | |
| SNP2 | SNP43 | SNP44 | SNP46 | | | |
| SNP2 | SNP43 | SNP45 | SNP46 | | | |
| SNP2 | SNP44 | SNP45 | SNP46 | | | |
| SNP3 | SNP6 | SNP43 | SNP44 | | | |
| SNP3 | SNP6 | SNP43 | SNP45 | | | |
| SNP3 | SNP6 | SNP43 | SNP46 | | | |
| SNP3 | SNP6 | SNP44 | SNP45 | | | |
| SNP3 | SNP6 | SNP44 | SNP46 | | | |
| SNP3 | SNP6 | SNP45 | SNP46 | | | |
| SNP3 | SNP43 | SNP44 | SNP45 | | | |
| SNP3 | SNP43 | SNP44 | SNP46 | | | |
| SNP3 | SNP43 | SNP45 | SNP46 | | | |
| SNP3 | SNP44 | SNP45 | SNP46 | | | |
| SNP6 | SNP43 | SNP44 | SNP45 | | | |
| SNP6 | SNP43 | SNP44 | SNP46 | | | |
| SNP6 | SNP43 | SNP45 | SNP46 | | | |
| SNP6 | SNP44 | SNP45 | SNP46 | | | |
| SNP43 | SNP44 | SNP45 | SNP46 | | | |
| SNP2 | SNP3 | SNP6 | SNP43 | SNP44 | | |
| SNP2 | SNP3 | SNP6 | SNP43 | SNP45 | | |
| SNP2 | SNP3 | SNP6 | SNP43 | SNP46 | | |
| SNP2 | SNP3 | SNP6 | SNP44 | SNP45 | | |
| SNP2 | SNP3 | SNP6 | SNP44 | SNP46 | | |
| SNP2 | SNP3 | SNP6 | SNP45 | SNP46 | | |
| SNP2 | SNP3 | SNP43 | SNP44 | SNP45 | | |
| SNP2 | SNP3 | SNP43 | SNP44 | SNP46 | | |
| SNP2 | SNP3 | SNP43 | SNP45 | SNP46 | | |
| SNP2 | SNP3 | SNP44 | SNP45 | SNP46 | | |
| SNP2 | SNP6 | SNP43 | SNP44 | SNP45 | | |
| SNP2 | SNP6 | SNP43 | SNP44 | SNP46 | | |
| SNP2 | SNP6 | SNP43 | SNP45 | SNP46 | | |
| SNP2 | SNP6 | SNP44 | SNP45 | SNP46 | | |
| SNP2 | SNP43 | SNP44 | SNP45 | SNP46 | | |
| SNP3 | SNP6 | SNP43 | SNP44 | SNP45 | | |
| SNP3 | SNP6 | SNP43 | SNP44 | SNP46 | | |
| SNP3 | SNP6 | SNP43 | SNP45 | SNP46 | | |
| SNP3 | SNP6 | SNP44 | SNP45 | SNP46 | | |
| SNP3 | SNP43 | SNP44 | SNP45 | SNP46 | | |
| SNP6 | SNP43 | SNP44 | SNP45 | SNP46 | | |
| SNP2 | SNP3 | SNP6 | SNP43 | SNP44 | SNP45 | |
| SNP2 | SNP3 | SNP6 | SNP43 | SNP44 | SNP46 | |
| SNP2 | SNP3 | SNP6 | SNP43 | SNP45 | SNP46 | |
| SNP2 | SNP3 | SNP6 | SNP44 | SNP45 | SNP46 | |
| SNP2 | SNP3 | SNP43 | SNP44 | SNP45 | SNP46 | |
| SNP2 | SNP6 | SNP43 | SNP44 | SNP45 | SNP46 | |
| SNP3 | SNP6 | SNP43 | SNP44 | SNP45 | SNP46 | |
| SNP2 | SNP3 | SNP6 | SNP43 | SNP44 | SNP45 | SNP46 |

a. Detection of Polymorphisms

Detection techniques for evaluating nucleic acids for the presence of a SNP involve procedures well known in the field of molecular genetics. Many, but not all, of the methods involve amplification of nucleic acids. Ample guidance for performing amplification is provided in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology (Ausubel et al., John Wiley & Sons, New York, 2003); Molecular Cloning: A Laboratory Manual (Sambrook & Russell, Cold Spring Harbor Press, Cold Spring Harbor, N.Y., 3^{rd} Ed, 2001). General methods for detection of single nucleotide polymorphisms is disclosed in Single Nucleotide Polymorphisms: Methods and Protocols, Pui-Yan Kwok, ed., 2003, Humana Press.

Although the methods typically employ PCR steps, other amplification protocols may also be used. Suitable amplification methods include ligase chain reaction (see, e.g., Wu & Wallace, Genomics 4:560-569, 1988); strand displacement assay (see, e.g. Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels may be used, such as QR-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993.

Oligonucleotides for amplification or other procedures may be synthesized using commercially available reagents and instruments. Methods of synthesizing oligonucleotides are well known in the art (see, e.g, Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066). Alternatively, oligonucleotides may be purchased through commercial sources. On some embodiments, the oligonucleotide may be detectably labeled, for example, with a fluorescent moiety, a radioactive moiety, a biotin moiety. In some embodiments, the oligonucleotide may be detectably labeled with a fluorescent moiety attached to the 5′-end of the oligonucleotide. In some embodiments, the oligonucleotide may further comprise a quencher moiety that quenches the fluorescent moiety when the oligonucleotide is intact or unbound.

Methods suitable for detection of the polymorphism are well known in the art. Suitable assays include allele-specific real time PCR, 5′-nuclease assays, template-directed dye-terminator incorporation, molecular beacon allele-specific oligonucleotide assays, assays employing invasive cleavage with Flap nucleases, allele-specific hybridization (ASH), array based hybridization, allele-specific ligation, primer extension, single-base extension (SBE) assays, sequencing, pyrophosphate sequencing, real-time pyrophosphate sequencing, sequence length polymorphism analysis, restriction length fragment polymorphisms (RFLP), RFLP-PCR, single-stranded conformational polymorphism (SSCP), PCR-SSCP, fragment sizing capillary electrophoresis, heteroduplex analysis, and mass array systems. Analysis of amplified sequences may be performed using various technologies such as microchips, fluorescence polarization assays, and matrix-assisted laser desorption ionization (MALDI) mass spectrometry. In a preferred embodiment, the polymorphism is genotyped using the Sequenom MassArray technology (www.sequenom.com).

b. Sample

Determination of the presence of a particular allele of a polymorphism is generally performed by analyzing a nucleic acid sample that is obtained from the subject to be analyzed. To determine genomic polymorphisms, the nucleic acid sample generally comprises genomic DNA. The nucleic acid may be isolated from a biological sample using methods commonly known in the art. A skilled artisan would appreciate that the method of isolation can and will vary depending on the nucleic acid to be isolated and the biological sample used. For more information, see Ausubel et al., 2003, or Sambrook & Russell, 2001. Commercially available DNA or RNA extraction kits or commercially available extraction reagents may be used to isolate the nucleic acid from the biological sample.

Non-limiting examples of suitable biological samples include fluid samples, biopsy samples, skin samples, and hair samples. Fluid samples may include blood, serum, saliva, tears, and lymph. Furthermore, a lymphoblastoid cell line may be derived from the subject. Nucleic acid may be isolated from a blood sample, a saliva sample, an epithelial sample, a skin sample, a hair sample, a lymphoblastoid cell line, or other biological sample commonly used in the art. Methods of collecting a biological sample from a subject are well known in the art. In particular, methods of collecting blood samples, saliva samples, epithelial samples, and skin samples are well known in the art.

c. Subject

Typically, the subject to be analyzed for risk of substance dependence is human. The subject may be male or female and of any racial or ethnic origin. In some embodiments, the subject may be Caucasian, Asian, African, Negro, Hispanic, Indian, Native American, or a combination thereof.

d. Substance Dependence

The method of the invention analyzes polymorphisms associated with substance dependence. The method may be used to determine the risk for dependence on substances such as alcohol, cocaine, heroin, methamphetamine, and prescription medications, such as opiods and central nervous system depressants. In a preferred embodiment, the substance dependence may be alcohol dependence.

### (II) Method for Determining the Response of a Subject to a Therapeutic Substance

Another aspect of the invention encompasses a method for determining the response of a subject to a therapeutic substance. The method comprises detecting in a sample from the subject the presence of at least one polymorphism in the CHRNA5-CHRNA3-CHRNB4 gene cluster and the CHRNA4 gene, wherein the polymorphisms have a correlation value (R²) of 0.7 or greater with each other. Accordingly, the presence of one allele of the polymorphism is associated with one response and the presence of another allele of the polymorphism is associated with another response.

In one embodiment, the polymorphism may be selected from those listed in Table A. In another embodiment, the polymorphism may be from a variant in positive linkage disequilibrium with the polymorphism selected from those listed in Table A. In another embodiment, a combination of polymorphisms may be selected from those listed in Table A. In another embodiment, the polymorphism may be from a variant in positive linkage disequilibrium with the polymorphism selected from those listed in Table B. In still another embodiment, the polymorphism may be selected from those listed in Table B. In an alternate embodiment, a combination of polymorphisms may be selected from those listed in Table B. Methods of detecting the polymorphism were described above in Section (I).

As shown in Example 3, one allele of SNP 6 is associated with increased levels of CHRNA5 mRNA and the other allele of SNP 6 is associated with decreased levels of CHRNA5 mRNA. Additionally, highly correlated SNPs display this same relationship. Consequently, the level of the alpha 5 subunit of the nicotinic acetylcholine receptor may also be altered accordingly in a subject. Thus, a subject with a particular allele of one of these polymorphisms may respond to a particular therapeutic substance that interacts, directly or indirectly, with a nicotinic acetylcholine receptor differently than a subject with an alternate allele of the polymorphism.

A subject with one allele of the polymorphism may have an altered sensitivity to the therapeutic substance relative to a subject with another allele of the polymorphism. Thus, the dosage of the therapeutic substance may be adjusted accordingly for each subject. Similarly, a subject with one allele of the polymorphism may have more or less adverse reactions to the therapeutic substance relative to a subject with another allele of the polymorphism. Those of skill in the art will appreciate other applications of this method.

Non-limiting examples of a therapeutic substance that may affect the activity of a nicotinic acetylcholine receptor include a nicotinic acetylcholine receptor agonist, such as acetylcholine, nicotine, carbamylcholine, methylcarbamylcholine, epibatidine, epiboxidine, and altinicline; a nicotinic acetylcholine receptor partial agonist, such as varenicline, isopronidine, tropisetron, cytsine, and imidacloprid; an acetylcholinesterase inhibitor; and a nicotinic acetylcholine receptor antagonist, such as bupropion, hexamethonium, mecamylamine, fluoxetine, and iptakalim.

### (III) Method for Treating Substance Dependence in a Subject

A further aspect of the invention provides a method for treating substance dependence in a subject. The method comprises administering to the subject an agent that alters the level of the alpha 5 subunit of the nicotinic acetylcholine receptor and/or alters the activity of the alpha 5 subunit of the nicotinic acetylcholine receptor. Altered levels or activity of the alpha 5 subunit of the nicotinic acetylcholine receptor may decrease the dependency of the subject's body for the substance, reduce the levels of substance intake, decrease the rewarding properties of the substance, and/or diminish substance seeking behavior. Agents that may alter the activity of a nicotinic acetylcholine receptor were listed above in Section (II).

The agent used to treat substance dependence may be administered to the subject locally or systemically. The administration may be oral, parenteral, by inhalation spray, intrapulmonary, rectal, intradermal, transdermal, or topical in dosage unit formulations containing conventional nontoxic pharmaceutically acceptable carriers, adjuvants, and vehicles as desired. The term parenteral as used herein includes subcutaneous, intravenous, intramuscular, intraarterial, intraperitoneal, intracochlear, or intrasternal injection, or infusion techniques. In a preferred embodiment, the agent used to treat substance dependence is administered orally.

### (IV) Method for Evaluating the Response of a Subject to a Substance Cessation Treatment

Still another aspect of the invention encompasses a method for evaluating the response of a subject to a substance cessation treatment. The method comprises determining the level of CHRNA5 messenger RNA in a sample from the subject that was collected before the start of the substance cessation treatment, and determining the level of CHRNA5 messenger RNA in a sample that was collected during or after the treatment. A change in the level of CHRNA5 messenger RNA after a period of treatment generally indicates that the subject is responding to the substance cessation treatment.

The subject and the sample were described above in Section (I). For this method, the nucleic acid sample typically comprises RNA.

Detection of CHRNA5 messenger RNA may be accomplished by a variety of methods. Additional information regarding the methods presented below may be found, for example, in Ausubel et al., 2003 or Sambrook & Russell, 2001. A person skilled in the art will know which parameters may be manipulated to optimize detection of the mRNA of interest.

Quantitative real-time PCR (qRT-PCR) may be used to measure the levels of a particular mRNA. In qRT-PCR, the RNA template is generally reverse transcribed into cDNA, which is then amplified via a PCR reaction. The amount of PCR product generated is followed cycle-by-cycle in real time, which ultimately allows for determination of the initial concentrations of mRNA or cDNA in the sample. The quantification may be relative or absolute. To measure the amount of PCR product, the reaction may be performed in the presence of a fluorescent dye, such as SYBR® Green (Invitrogen, Carlsbad, Calif.), which binds to double-stranded DNA. The reaction may also be performed in the presence of a fluorescent reporter probe or primer/probe that is specific for the DNA being amplified. Non-limiting examples of fluorescent reporter probes include TaqMan® probes (Applied Biosystems, Foster City, Calif.) and secondary structure probes, such as molecular beacons and Scorpion primer/probes. To minimize errors and reduce any sample-to-sample variation, qRT-PCR is typically performed using an external and/or an internal standard. Suitable internal standards include, but are not limited to, mRNAs for the housekeeping genes glyceraldehyde-3-phosphate-dehydrogenase (GAPDH), beta-actin, or 18S rRNA.

Gene expression may also be measured using a nucleic acid microarray. In this method, single-stranded nucleic acids (e.g., cDNAs, oligonucleotides) are plated, or arrayed, on a microchip substrate. The arrayed sequences are then hybridized with specific DNA probes generated from the samples of interest. Fluorescently labeled cDNA probes may be generated through incorporation of fluorescently labeled deoxynucleotides by reverse transcription of RNA samples of interest. The probes are hybridized to the immobilized nucleic acids on the microchip under highly stringent conditions. After stringent washing to remove non-specifically bound probes, the chip is scanned by confocal laser microscopy or by another detection method, such as a CCD camera. Quantitation of hybridization of each arrayed element allows for assessment of corresponding mRNA abundance. Microarray analysis may be performed by commercially available equipment, following manufacturer's protocols, such as by using the Affymetrix GenChip technology, or Incyte's microarray technology.

Gene expression may also be measured using Luminex microspheres, in which molecular reactions take place on the surface of microscopic polystyrene beads. The beads are internally color-coded with fluorescent dyes, such that each bead has a unique spectral signature (of which there are up to 100). The surface of each bead is tagged with a specific oligonucleotide that can bind the target (i.e., mRNA) of interest. The target, in turn, is often attached to a reporter, which is also fluorescently tagged. Hence, there are two sources of color, one from the bead and the other from the reporter molecule. The small size/surface area of the beads and the three dimensional exposure to the targets allows for nearly solution-phase kinetics during the binding reaction. The captured targets are detected by high-tech fluidics based upon flow cytometry in which lasers excite the internal dyes that identify each bead and also any reporter dye captured during the assay.

Levels of mRNA may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe that is complementary to the mRNA of interest. After washing to remove the non-specifically bound probe, the hybridized labeled species are detected using techniques well known in the art. The probe may be labeled with a radioactive element, a chemical that fluoresces when exposed to ultraviolet light, a tag that is detected with an antibody, or an enzyme that catalyses the formation of a colored or a fluorescent product

Nuclease protection assays may also be used to monitor the levels of mRNA. In nuclease protection assays, an antisense probe hybridizes in solution to the mRNA of interest. The antisense probe may be labeled with an isotope, a fluorophore, an enzyme, or another tag. Following hybridization, nucleases are added to degrade the single-stranded, unhybridized probe and mRNA. An acrylamide gel is used to separate the remaining protected double-stranded fragments, which are then detected using techniques well known in the art.

### (V) Method for Predicting the Response of a Subject to a Substance Cessation Treatment

Another aspect of the invention encompasses a method for predicting the response of a subject to a substance cessation treatment. The method comprises detecting in a sample from the subject the presence of at least one polymorphism in the CHRNA5-CHRNA3-CHRNB4 gene cluster and the CHRNA4 gene, wherein the polymorphisms have a correlation value (R²) of 0.7 or greater with each other. The presence of one allele of the polymorphism is associated with a positive response to the substance cessation treatment and the presence of another allele of the polymorphism is associated with a negative response to the treatment. The relevant polymorphisms in the CHRNA5-CHRNA3-CHRNB4 gene cluster and the CHRNA4 gene, and the methods of detecting polymorphisms were described above in Section (I).

### (VI) Kit for Genotyping a Subject

In yet another aspect, the invention provides a kit for genotyping a subject for at least one polymorphism in the CHRNA5-CHRNA3-CHRNB4 gene cluster and the CHRNA4 gene. The kit comprises at least one oligonucleotide that distinguishes between two alleles of one of the relevant polymorphisms. In one embodiment, the kit may comprise oligonucleotide(s) that distinguish between the alleles of polymorphisms selected from the group consisting SNP 1, SNP 2, SNP 3, SNP 6, SNP 11, SNP 12, SNP 15, SNP 17, SNP 19, SNP 23, SNP 30, SNP 31, SNP 32. SNP 40, SNP, 41, SNP 42, SNP 43, SNP 44, SNP 45, SNP 46 and a combination thereof. In another embodiment, the kit may comprise a) at least one oligonucleotide that distinguishes between the long allele and the short allele of SNP 6; b) at least one oligonucleotide that distinguishes between the A allele and the T allele of SNP 11; c) at least one oligonucleotide that distinguishes between the A allele and the G allele of SNP 12; d) at least one oligonucleotide that distinguishes between the C allele and the G allele of SNP 15; e) at least one oligonucleotide that distinguishes between the C allele and the T allele of SNP 19; f) at least one oligonucleotide that distinguishes between the G allele and the A allele of SNP 40; g) at least one oligonucleotide that distinguishes between the G allele and the A allele of SNP 41, h) at least one oligonucleotide that recognizes the T allele of SNP 42; i) SNP 43, wherein the presence of A rather than G is associated with increased risk for substance dependence, j) SNP 44, wherein the presence of A rather than G is associated with increased risk for substance dependence, k) SNP 45, wherein the presence of T rather than C is associated with increased risk for substance dependence, l) SNP 46, wherein the presence of T rather than C is associated with increased risk for substance dependence, or m) a combination thereof.

The oligonucleotide or oligonucleotides of the kit may be used to detect the polymorphisms using any of the methods that were detailed above in Section (I). The oligonucleotide may be exactly complementary to the sequence of interest. Alternatively, the oligonucleotide may be substantially complementary to the sequence of interest. “Substantially complementary” refers to sequences that are complementary except for minor regions of mismatch. Typically, the total number of mismatched nucleotides over a hybridizing region is not more than 3 nucleotides for sequences about 15 nucleotides in length. Conditions under which only exactly complementary nucleic acid strands will hybridize are referred to as “stringent” hybridization conditions. Stable duplexes of substantially complementary nucleic acids can be achieved under less stringent hybridization conditions. Those skilled in the art of nucleic acid technology are able to determine duplex stability empirically considering a number of variables including, for example, the length and base pair concentration of the oligonucleotides, ionic strength, and incidence of mismatched base pairs.

Stringent conditions, under which an oligonucleotide will hybridize only to the exactly complementary target sequence, are well known in the art (see, e.g. Ausubel et al., 2003 or Sambrook & Russell, 2001). Stringent conditions are sequence dependent and will be different in different circumstances. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the base pairs have dissociated. Relaxing the stringency of the hybridizing conditions will allow sequence mismatches to be tolerated; the degree of mismatch tolerated may be controlled by suitable adjustment of the hybridization conditions.

The oligonucleotides in the kit optionally may be detectably labeled, for example, with a fluorescent moiety, a radioactive moiety, a biotin moiety, and/or a quencher moiety. In some embodiments, the kit may further comprise a thermostable polymerase. In other embodiments, the kit may further comprise a reaction buffer. The reaction buffer may comprise a buffering agent, such as Tris buffers, MOPS, HEPES, Bicine, Tricine, TES, or PIPES, a monovalent cation, such as potassium, sodium, or lithium, a divalent cation, such as magnesium and/or manganese, and/or a detergent, such as Tween 20, and Nonidet NP40. In still other embodiments, the kit may further comprise a mixture of deoxynucleotide triphosphates.

### DEFINITIONS

To facilitate understanding of the invention, a number of terms are defined below.

The term “allele” refers to one of two or more different nucleotide sequences that occur at a specific locus, or two or more different polypeptides encoded by such a locus. The term “risk allele” refers to the allele that positively correlates with substance dependence, and the term “protective allele” refers to the allele that negatively correlates with substance dependence or is protective for substance dependence.

As used herein, “substance dependence” refers to the body's physical need, or addiction, to a specific substance. Stopping the use of the substance may result in a specific withdrawal syndrome.

The term “linkage disequilibrium” or “LD” as used herein, refers to alleles at different loci that are not associated at random, that is, not associated in proportion to their frequencies. If the alleles are in positive linkage disequilibrium, then the alleles occur together more often than expected, assuming statistical independence. Conversely, if the alleles are in negative linkage disequilibrium, then the alleles occur together less often than expected, assuming statistical independence.

A “locus” is a chromosomal location or position. A “gene locus” is a specific chromosome location in the genome of a species where a specific gene can be found.

The term “oligonucleotide,” as used herein, refers to a molecule comprising two or more nucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, or phosphorodiamidate bonds.

A “polymorphism” is a locus that is variable; that is, the nucleotide sequence at a polymorphic locus has more than one version or allele within a population. An example of a polymorphism is a single nucleotide polymorphism (SNP), which is a polymorphism at a single nucleotide position in a genome (i.e., the nucleotide at the position varies between individuals or populations). Nucleotide polymorphisms may occur at any region of a gene, that is, in the promoter region, an intron, or an exon. In some instances, the polymorphism results in a change in the protein sequence. The change in protein sequence may affect protein function or may not.

The CHRNA5-CHRNA3-CHRNB4 gene cluster refers to a cluster of genes that code for the alpha 5 subunit of the nicotinic acetylcholine receptor, the alpha 3 subunit of the nicotinic acetylcholine receptor, and the beta 4 subunit of the nicotinic acetylcholine receptor, respectively. The gene cluster is located on the long arm of chromosome 15 and is about 115 kbp in length. The CHRNA4 gene codes for the alpha 4 subunit of the nicotinic acetylcholine receptor; the gene is located on the long arm of chromosome 20. Within the context of this invention, the CHRNA5-CHRNA3-CHRNB4 genes and the CHRNA4 gene designate all CHRNA5, CHRNA3, CHRNB4, and CHRNA4 gene sequences or products in a cell or organism, including CHRNA5, CHRNA3, CHRNB4, and CHRNA4 coding sequences, CHRNA5, CHRNA3, CHRNB4, and CHRNA4 non-coding sequences (e.g., introns), CHRNA5, CHRNA3, CHRNB4, and CHRNA4 regulatory sequences controlling transcription, translation, and/or stability (e.g., promoter, enhancer, terminator, etc.), as well as all corresponding expression products, such as CHRNA5 mRNA, CHRNA3 mRNA, CHRNB4, and CHRNA4 mRNA and nAChRα5, nAChRα3, nAChRβ4, and nAChRα4 polypeptides (including pre-proteins and mature proteins). The CHRNA5-CHRNA3-CHRNB4 gene cluster and the CHRNA4 gene also comprise surrounding sequences of the CHRNA5, CHRNA3, CHRNB4, and CHRNA4 genes, including polymorphisms that are in linkage disequilibrium with polymorphisms located in the CHRNA5, CHRNA3, CHRNB4, and CHRNA4 genes.

As used herein, the acronym “SNP” refers to simple genetic polymorphisms that are listed in the public database dbSNP (www.ncbi.nlm.nih.gov/SNP/). The simple genetic polymorphisms include both single base nucleotide substitutions (SNPs) and short deletion and insertion polymorphisms.

As various changes could be made in the above compounds, complexes, and methods without departing from the scope of the invention, it is intended that all matter contained in the above description and in the examples presented below, shall be interpreted as illustrative and not in a limiting sense. All of the patent documents and the other references cited herein are hereby incorporated by reference in their entirety.

### EXAMPLES

The following examples illustrate various embodiments of the invention.

### Example 1

### Genetic Variants in the CHRNA5-CHRNA3-CHRNB4 Gene Cluster are Associated with Alcohol Dependence in the Collaborative Study on the Genetics of Alcoholism Dataset

A comprehensive genome wide association study and a candidate gene study using nicotine dependent smokers as cases and non-dependent smokers as controls demonstrated significant association between several genetic variants in nicotinic acetylcholine receptors (nAChR) and nicotine dependence (Bierut et al., Hum Mol Genet 2007, 16:24-35; Saccone et al, Hum Mol Genet 2007, 16:36-49). Since the CHRNA5, CHRNA3, and CHRNB4 genes, which encode the α5, α3, and β4 subunits of nAChR, respectively, cluster together on chromosome 15q, a comprehensive association analysis was performed with this gene cluster in the Collaborative Study on the Genetics of Alcoholism (COGA) families to investigate the role of genetic variants in these three nAChRs in risk for alcohol dependence.

Study subjects. Alcohol-dependent probands, defined by meeting lifetime criteria for both DSM-IIIR alcohol dependence (Diagnostic and Statistical Manual of Mental Disorders, 3^{rd} ed (revised), 1987, American Psychiatric Press, Washington, D.C.) and Feighner-criteria for definite alcoholism (Feighner et al., Arch Gen Psychiatry 1972, 26:57-63) were systematically recruited from alcohol-treatment units. Families in which two additional first-degree relatives also met lifetime criteria for alcohol dependence were invited to participate in the genetic protocol. A total of 262 families including 2309 individuals were selected for the genetic study and an average of 4.6 alcohol-dependent individuals per pedigree were genotyped (see www.niaaagenetics.org/coga_instruments/resources.html). Among these pedigrees, 298 individuals from 35 pedigrees are African American and 8 pedigrees are of mixed ancestry (by self-report).

All subjects were assessed using the Semi-Structured Assessment for the Genetics of Alcoholism (SSAGA) (Bucholz et al. J Stud Alcohol 1994, 55:149-158; Hesselbrock et al., Addiction 1999, 94:1361-1370). Affected individuals were those who were alcohol dependent by DSM-IV criteria. When multiple interviews were available, consistency in all interviews was required for case/control status. Unaffected individuals were those who drank but had no more than two DSM-IV symptoms of alcohol dependence and were not dependent on any illicit substance.

SNP assays. The Single Nucleotide Polymorphism database (dbSNP) (www.ncbi.nlm.nih.gov/SNP/) was used to identify single nucleotide polymorphisms (SNPs) within and flanking the CHRNA5, CHRNA3 and CHRNB4 genes on the long arm of chromosome 15. Sequenom MassArray technology (www.sequenom.com), comprising homogenous MassEXTEND (hME) or iPLEX assays, was used for genotyping. PCR primers, termination mixes, and multiplexing capabilities were determined with Sequenom MassARRAY Assay Designer software v3.1.2.2. Standard procedures were used to amplify PCR products; unincorporated nucleotides were deactivated with shrimp alkaline phosphatase. A primer extension reaction was then carried out with the mass extension primer and the appropriate termination mix (hME) or terminator (iPLEX). The primer extension products were then cleaned with resin and spotted onto a silicon SpectroChip. The chip was scanned with a mass spectrometry workstation (Bruker Daltonics Inc., Billerica, Mass.), and the resulting genotype spectra were analyzed with the Sequenom SpectroTYPER software v3.4. Call rates greater than 90% and HWE p value >0.05 were set as quality control measures. For the 22-bp insertion/deletion (in/del) polymorphism (rs3841324), PCR primers were selected using the MacVector 6.5.3 program (Accelrys) to yield a 132-bp or 154-bp genomic fragment containing the indel. The nonsynonymous coding SNP in exon 5, rs16969968, originally identified by sequencing 40 individuals from COGA families, was genotyped using an RFLP assay with Taq1 restriction enzyme. Genotypes of SNP6/rs3841324 and SNP19/rs16969968 were detected by electrophoresis on a 2% agarose gel.

Statistical analyses. Linkage disequilibrium (LD) between markers was computed using the program Transmit (Martin et al. Am J Hum Genet 2000 67:146-154). The family-based association test (FBAT) (Laird et al., Genet Epidemiol 2000, 19 (Suppl 1):536-42; Horvath et al. Eur J Hum Genet 2001, 9:301-306) was used to examine association between the SNPs and alcohol dependence, defined by DSM-IV criteria. FBAT builds on the original TDT method (Spielman et al., Am J Hum Genet 1993, 52:506-516) in which alleles transmitted to affected offspring are compared with the expected distribution of alleles among offspring. In particular, the method puts tests of different genetic models, tests of different sampling designs, tests involving different disease phenotypes, tests with missing parents, and tests of different null hypotheses all into the same framework. Similar in spirit to a classical TDT test, the approach compares the genotype distribution observed in the cases to its expected distribution under the null hypothesis, with the null hypothesis being no linkage and no association, or no association in the presence of linkage. Here, the expected distribution was derived using Mendel's law of segregation and conditioned on the sufficient statistics for any nuisance parameters under the null. Because conditioning eliminates all nuisance parameters, the technique avoids confounding due to model misspecification as well as admixture or population stratification.

Results. Forty-four single nucleotide polymorphisms (SNPs) and an indel (insertion/deletion) within and flanking this cluster of genes encoding these nAChRs were genotyped (FIG. 1A). Each of the SNPs was in Hardy-Weinberg equilibrium in the founders. Three SNPs that had a minor allele frequency (MAF) less than 5% were removed from analyses. Using pair-wise linkage disequilibrium analysis, three groups of highly correlated variants tagged by three putative functional polymorphisms were observed, a 22 by indel (SNP 6/rs3841324) in the promoter region of the CHRNA5 gene, a missense mutation (SNP 16/rs16969968) in exon 5 of the CHRNA5 gene, and a SNP (SNP 18/rs578776) in the 3′UTR of the CHRNA3 gene, respectively (Table 1). The data shown are from the standard analysis with age and gender as covariates.

TABLE 1
FBAT analysis of SNPs in the cluster of CHRNA5-
CHRNA3-CHRNB4 genes with alcohol dependence
in the COGA European American dataset.

| | dbSNP | Chromosome | | | | |
|---|---|---|---|---|---|---|
| SNP | reference | Position | Allele | MAF | Nf | P value |
| 1 | rs1979906* | 76629344 | A/G | 0.44 | 171 | 0.008 |
| 2 | rs880395* | 76631411 | A/G | 0.43 | 162 | 0.088 |
| 3 | rs7164030* | 76631716 | A/G | 0.44 | 149 | 0.113 |
| 4 | rs905739\hat{ } | 76632165 | C/T | 0.22 | 120 | 0.179 |
| 5 | rs2036527# | 76638670 | C/T | 0.33 | 145 | 0.166 |
| 6 | rs3841324* | 76644877 | L/S | 0.44 | 172 | 0.005 |
| 7 | rs503464\hat{ } | 76644951 | A/T | 0.22 | 133 | 0.192 |
| 8 | rs684513\hat{ } | 76645455 | C/G | 0.20 | 116 | 0.277 |
| 9 | rs667282\hat{ } | 76650527 | C/T | 0.23 | 136 | 0.352 |
| 10 | rs17486278# | 76654537 | A/C | 0.32 | 138 | 0.078 |
| 11 | rs601079* | 76656634 | A/T | 0.43 | 180 | 0.026 |
| 12 | rs680244* | 76658343 | A/G | 0.44 | 175 | 0.003 |
| 13 | rs621849* | 76659916 | A/G | 0.44 | 175 | 0.004 |
| 14 | rs569207\hat{ } | 76660174 | A/G | 0.23 | 133 | 0.121 |
| 15 | rs692780* | 76663560 | C/G | 0.38 | 172 | 0.013 |
| 16 | rs16969968# | 76669980 | A/G | 0.34 | 154 | 0.177 |
| 17 | rs514743* | 76671282 | A/T | 0.38 | 176 | 0.117 |
| 18 | rs578776\hat{ } | 76675455 | C/T | 0.28 | 146 | 0.810 |
| 19 | rs6495307* | 76677376 | C/T | 0.43 | 172 | 0.010 |
| 20 | rs12910984\hat{ } | 76678682 | A/G | 0.23 | 132 | 0.345 |
| 21 | rs1051730# | 76681394 | C/T | 0.32 | 147 | 0.016 |
| 22 | rs3743078\hat{ } | 76681814 | C/G | 0.24 | 137 | 0.671 |
| 23 | rs3743077* | 76681951 | A/G | 0.42 | 176 | 0.080 |
| 24 | rs938682\hat{ } | 76683602 | C/T | 0.23 | 133 | 0.602 |
| 25 | rs11637630\hat{ } | 76686774 | A/G | 0.23 | 132 | 0.228 |
| 26 | rs7177514\hat{ } | 76694461 | C/G | 0.24 | 128 | 0.589 |
| 27 | rs6495308\hat{ } | 76694711 | C/T | 0.24 | 132 | 0.870 |
| 28 | rs8042059\hat{ } | 76694914 | A/C | 0.23 | 131 | 0.851 |
| 29 | rs8042374\hat{ } | 76695087 | A/G | 0.23 | 121 | 0.432 |
| 30 | rs3743075* | 76696507 | A/G | 0.39 | 175 | 0.207 |
| 31 | rs3743073* | 76696594 | A/C | 0.39 | 175 | 0.199 |
| 32 | rs1878399* | 76699058 | C/G | 0.43 | 175 | 0.061 |
| 33 | rs17487223# | 76711042 | C/T | 0.35 | 144 | 0.065 |
| 34 | rs950776 | 76713073 | C/T | 0.35 | 164 | 0.872 |
| 35 | rs11636605 | 76715933 | A/G | 0.22 | 112 | 0.975 |
| 36 | rs9920506 | 76718112 | A/G | 0.19 | 102 | 0.716 |
| 37 | rs3813567 | 76721606 | C/T | 0.22 | 125 | 0.698 |
| 38 | rs17487514 | 76740840 | C/T | 0.30 | 156 | 0.052 |
| 39 | rs1996371 | 76743861 | A/G | 0.39 | 154 | 0.155 |

*represents SNPs that are highly correlated (r2 ≧ 0.7) with a 22-bp indel (SNP 6/rs3841324) in CHRNA5.
#represents SNPs that are highly correlated with a missense mutation (SNP 16/rs16969968) in exon 5 of CHRNA5.
\hat{ }represents SNPs that are highly correlated with a SNP (SNP 18/rs578776) that maps to the 3′UTR of CHRNA3.
MAF = minor allele frequency; Nf = number of informative families.

Using FBAT, a strong association for a 22 by indel (SNP 6/rs3841324) was detected in the promoter region of CHRNA5 with alcohol dependence in analyses adjusted for age and gender (Table 1, FIG. 1B). Several SNPs that are highly correlated (r²≧0.7) with SNP 6/rs3841324 also show significant association with alcohol dependence. These associated SNPs include one SNP upstream of the CHRNA5 gene, 4 intronic SNPs in the CHRNA5 gene, and 2 intronic SNPs in the CHRNA3 gene (Table 1). To determine whether this association was driven by nicotine dependence, the association with habitual smoking was also analyzed as a covariate and it was found that the association of this promoter polymorphism with alcohol dependence was independent of smoking status.

In contrast, no association was observed between alcohol dependence and either of the SNPs previously reported to be associated with nicotine dependence: the missense mutation (SNP 16/rs16969968) in CHRNA5 and SNP 18/rs578776 located within the 3′UTR of CHRNA3 (Saccone et al., 2007; Bierut et al. New Engl J Med, unpublished). A similar pattern of association was seen for all SNPs across the gene cluster in affected only analyses and in analyses without covariates.

### Example 2

### Replication Study with the Family Study of Cocaine Dependence (FSCD)

To further examine the genetic contribution of SNPs in this gene cluster with respect to risk for alcohol dependence, SNP 6/rs38413234 and 10 other SNPs in linkage disequilibrium with SNP 6/rs38413234 (in European Americans) were genotyped in an independent dataset from the FSCD.

Study subjects. Unrelated cases and matched unrelated controls within the candidate-gene study of the FSCD were used for this study. Cocaine dependent subjects were recruited from publicly and privately funded inpatient and outpatient chemical dependency treatment centers in the St. Louis area. Eligibility requirements included meeting DSM-IV criteria for cocaine dependence, being 18 years of age or older, speaking fluent English, and having a full sibling within five years of their age who was willing to participate in the family-arm of the study. Control subjects were recruited through driver's license records maintained by the Missouri Family Registry at Washington University in St. Louis for research purposes. Controls were matched to cocaine dependent subjects based on age, ethnicity, gender, and zip code. If subjects were dependent on alcohol or drugs, including nicotine, they were excluded from the control group. Subjects were also excluded if they had never used alcohol because such individuals are considered phenotypically unknown. The project was approved by the Washington University IRB and all subjects provided informed consent. All participants completed a modified version of the SSAGA (Bucholz et al., 1994; Hesselbrock et al., 1999).

SNP assays. The assays were as described above in Example 1.

Statistical analyses. Logistic regression (Hosmer, Applied Logistic Regression, Wiley, New York, 1989) was used to examine the association between the SNPs and DSM-IV alcohol dependence. For analysis, those cases who were comorbid for DSM-IV alcohol and cocaine dependence were selected and compared with all of the study controls. This subset included 451 unrelated individuals of European-American descent (207 alcohol-dependent cases and 244 controls) and 424 unrelated individuals of African-American descent (185 alcohol-dependent cases and 239 controls). Separate logistic regression models were run for the European and African American subjects as well as a combined analysis that incorporated all samples and included race as a covariate. Three logistic regression models were examined for each SNP to test for additive effects and evidence of dominant or recessive modes of inheritance. The additive effect of a SNP was modeled using an ordinal measure of the number of copies of the risk allele. The dominant and recessive effects of a SNP were modeled using dichotomous indicator variables. For each SNP, the model with the strongest association with DSM-IV alcohol dependence, based on the adjusted odds ratio and the magnitude of the corresponding p-value, is reported in Table 2.

Results. Using logistic regression analysis, the association between each of these SNPs and alcohol dependence was confirmed in the subjects of European descent in the FSCD dataset (Table 2). The data are shown with age and sex as covariates. In the African American subset, three SNPs that had the same minor allele frequencies as in European Americans also showed significant association with alcohol dependence. In contrast, the minor alleles of (SNP 6/rs3841324 and SNP 23/rs3743077 were less common in African Americans (Table 2): SNP 23/rs3743077 still showed association with alcohol dependence but SNP 6/rs3841324 did not. When the combined European and African American dataset was analyzed, however, a stronger association was detected than in the European American dataset alone for 8 of the 11 SNPs including SNP 6/rs3841324 (Table 2).

TABLE 2
Logistic regression analysis of selected SNPs with alcohol dependence in FSCD dataset.

| | | European Americans | | | African Americans | | | Combined Dataset | |
|---|---|---|---|---|---|---|---|---|---|
| | dbSNP | | | p- | | | p- | | p- |
| SNP | reference | MAF | OR (CI) | value | MAF | OR (CI) | value | OR (CI) | value |
| 2 | rs880395 | 0.46 | 1.32 (1.02, 1.71) | 0.037 | 0.23 | 1.25 (0.89, 1.76) | 0.193 | 1.29 (1.05, 1.58) | 0.015 |
| 3 | rs7164030 | 0.47 | 1.35 (1.04, 1.75) | 0.023 | 0.23 | 1.24 (0.88, 1.74) | 0.221 | 1.30 (1.06, 1.60) | 0.012 |
| 6 | rs3841324 | 0.47 | 1.35 (1.04, 1.76) | 0.022 | 0.22 | 1.24 (0.88, 1.75) | 0.219 | 1.31 (1.07, 1.62) | 0.010 |
| 11 | rs601079 | 0.46 | 1.34 (1.03, 1.74) | 0.028 | 0.43 | 1.37 (1.02, 1.84) | 0.034 | 1.35 (1.11, 1.64) | 0.002 |
| 12 | rs680244 | 0.46 | 1.35 (1.04, 1.75) | 0.023 | 0.43 | 1.36 (1.02, 1.82) | 0.039 | 1.35 (1.11, 1.64) | 0.002 |
| 15 | rs692780 | 0.40 | 1.37 (1.05, 1.78) | 0.022 | 0.24 | 1.47 (1.04, 2.08) | 0.029 | 1.41 (1.14, 1.74) | 0.002 |
| 19 | rs6495307 | 0.45 | 1.30 (1.00, 1.69) | 0.046 | 0.42 | 1.47 (1.09, 1.97) | 0.012 | 1.37 (1.13, 1.67) | 0.001 |
| 23 | rs3743077 | 0.46 | 1.34 (1.03, 1.74) | 0.027 | 0.14 | 1.18 (0.75, 1.86) | 0.471 | 1.38 (1.10, 1.72) | 0.005 |
| 30 | rs3743075 | 0.40 | 1.29 (0.99, 1.68) | 0.058 | 0.45 | 1.10 (0.83, 1.47) | 0.511 | 1.20 (0.99, 1.46) | 0.063 |
| 31 | rs3743073 | 0.41 | 1.32 (1.02, 1.73) | 0.038 | 0.45 | 1.09 (0.82, 1.45) | 0.561 | 1.21 (1.00, 1.47) | 0.053 |
| 32 | rs1878399 | 0.46 | 1.32 (1.02, 1.71) | 0.038 | 0.26 | 1.09 (0.79, 1.51) | 0.601 | 1.22 (1.00, 1.50) | 0.052 |

MAF = minor allele frequency; OR = odd ratio; CI = confidence interval.

### Example 3

### Allelic Differences in Expression of the CHRNA5 Gene in Human Frontal Cortex

To determine whether the SNPs associated with alcohol dependence have a direct effect on gene expression, the levels of CHRNA5 mRNA were analyzed in human brain tissue obtained from the Alzheimer's Disease Research Center (ADRC) of Washington University in St. Louis.

Gene expression analyses. Postmortem brain tissues derived from frontal cortex of 48 unrelated, non-demented adults were obtained from the brain bank at the Alzheimer's Disease Research Center (ADRC) of Washington University in St. Louis (alzheimer.wustl.edu/). DNA and total RNA was extracted from brain tissues using Qiagen's DNeasy Blood & Tissue Kit and RNeasy Lipid Tissue Kit (www.qiagen.com), respectively. A cDNA library was prepared from total RNA using the High Capacity cDNA Archive Kit (www.appliedbiosystems.com).

Genomic DNA from all subjects was genotyped for the promoter polymorphism, SNP 6/rs3841324 as described above in Example 1. Gene expression level was analyzed by real-time PCR using an ABI-7500 real-time PCR system. A TaqMan assay (Hs00181248_m1, ABI) was used for quantifying the expression level of the CHRNA5 mRNA. Primers and TaqMan probe for the reference gene, GAPDH were designed over exon-exon boundaries using the Primer Express 3 (ABI) program.

Each real-time PCR run included within-plate duplicates and each experiment was performed twice for each sample. Correction for sample-to-sample variation was done by simultaneously amplifying GAPDH as a reference. Real-time data were analyzed using the comparative Ct method (Muller et al., Biotechniques 2002, 32:1372-1374, 1376, 1378-1379). The Ct values of each sample were normalized with the Ct value for the housekeeping gene, GADPH and were corrected for the PCR efficiency of each assay, although the efficiency of all reactions was close to 100%. Only the samples with a standard error <0.15 were analyzed. Non-parametric Mann-Whitney U statistic was used to test for evidence of differential expression in samples homozygous for the long allele and samples homozygous for the short allele.

Results. Forty-eight samples of genomic DNA were genotyped with SNP6/rs3841324. The genotypes were in Hardy-Weinberg equilibrium. CHRNA5 468591.1 mRNA expression was examined in 9 samples homozygous for the long allele, 5 samples homozygous for the short allele, and 9 heterozygous samples. Subjects homozygous for the long allele (i.e., the reference allele) of SNP 6/rs3841324 showed a 2.8-fold reduction (LL=2.04±1.57; SS=5.74±1.62; P=0.007) in CHRNA5 mRNA expression compared to subjects homozygous for the short allele (FIG. 2). Heterozygotes for SNP 6/rs3841324 showed lower expression levels for CHRNA5 mRNA, compared with homozygotes for the short allele (LS=2.76±1.78; SS=5.74±1.62; P=0.012). However, no significant differences were found between heterozygotes and homozygotes for the long allele (FIG. 2).

This observation was further validated using an independent dataset and methodology. For this, an association between variability in CHRNA5 mRNA expression and alcohol dependence was tested in lymphoblastoid cell lines derived from CEPH families using the Affymetrix HG Focus panel (Genetic Analysis Workshop 15; www.gaworkshop.org/gaw15.htm) (Morley et al., Nature 2004, 430:743-747; Cheung et al., Nature 2005, 437:1365-1369). Because SNP 6/rs3841324 was not included in GAW15 dataset, genotypes were retrieved for the highly correlated SNP 12/rs680244 (r2=0.82 in European Americans) from the HapMap database. The difference in mRNA expression was examined in subjects with different genotypes at SNP 12/rs680244 in 14 genotyped trios. Using SOLAR VC quantitative analysis of the CHRNA5 mRNA levels with additive genetic effects, significant differences were detected in expression in subjects of different genotypes (p=0.04). This SNP accounted for approximately 10% of the variance in CHRNA5 gene expression in this system.

In conclusion, replicated evidence has been provided of association between multiple SNPs within the CHRNA5 and CHRNA3 genes and alcohol dependence. Furthermore, it was demonstrated that the risk allele of these SNPs is associated with higher CHRNA5 mRNA levels in human frontal cortex.

### Example 4

### Effect of Genetic Variants Associated with Substance Dependence on mRNA Expression

There is extensive linkage disequilibrium across the CHRNA5, CHRNA3, and CHRNB4 gene cluster, making it difficult to determine which gene(s) affects risk for substance dependence. In this study, the methods used in Example 3 were extended to examine whether the variants associated with substance dependence have a direct effect on CHRNA5, CHRNA3, and CHRNB4 gene expression.

Gene expression analysis. Postmortem brain tissue from the frontal cortex of 48 unrelated, non-demented elderly European Americans were obtained from the Alzheimer's Disease Research Center at Washington University (alzheimer.wustl.edu/). A second set of frontal cortex samples derived from 34 unrelated, non-alcoholic European Australians were obtained from the Australian Brain Donor Program, Sydney, Australia (www.braindonors.org/). Each genomic DNA sample was genotyped with the SNPs listed in Table 3. Total mRNA expression levels for CHRNA5, CHRNA3 and CHRNB4 were assessed by quantitative real-time PCR with an ABI-7500 system. Real-time data were analyzed using the comparative Ct method. Logistic regression was used to test for evidence of differential expression in samples of different genotype.

TABLE 3
Association of mRNA expression with variants
associated with alcohol dependence.

| | P values | | |
|---|---|---|---|
| SNP | CHRNA5 | CHRNA3 | CHRNB4 |
| rs3841324 | <.0001 | 0.031 | 0.795 |
| rs588765 | <.0001 | 0.011 | 0.616 |
| rs601079 | <.0001 | 0.011 | 0.630 |
| rs569207 | 0.006 | 0.021 | 0.820 |
| rs637137 | 0.008 | 0.018 | 0.791 |
| rs16969968 | 0.083 | 0.793 | 0.755 |
| rs578776 | 0.133 | 0.044 | 0.454 |
| rs3743078 | 0.007 | 0.033 | 0.913 |
| rs11637630 | 0.008 | 0.021 | 0.706 |
| rs17487223 | 0.168 | 0.742 | 0.550 |
| rs1996371 | 0.836 | 0.778 | 0.097 |

Results. Quantitative real-time PCR analysis demonstrates that variants rs3841324, rs588765, and rs601079, associated with alcohol dependence, affect CHRNA5 mRNA expression in both datasets (p<0.0001; Table 3). Subjects homozygous for the minor (S) allele of the SNP rs3841324 show 2.9-fold increase in CHRNA5 mRNA levels in frontal cortex (FIG. 3), but only moderate effect on CHRNA3 mRNA levels (FIG. 4), and no effect on CHRNB4 (FIG. 5) mRNA levels. Similarly, subjects homozygous for the minor T allele of the SNP rs588765 show an increase in CHRNA5 mRNA levels in frontal cortex (FIG. 6), a moderate decrease in CHRNA3 mRNA levels (FIG. 7), and no effect on CHRNB4 (FIG. 8) mRNA levels.

The genetic variants associated with increased risk for nicotine dependence, including the missense variant rs16969968, do not affect mRNA expression of any of the genes in this cluster (Table 3). Several of the variants associated with reduced risk for nicotine dependence are also associated with CHRNA5 mRNA levels. However, these SNPs (rs569207, rs637137, 3743078, and rs11637630) are no longer significant once rs3841324 or rs588765 is included in a stepwise discriminant analysis of CHRNA5 mRNA levels suggesting that this association is driven by the linkage disequilibrium with variants that show association with alcohol dependence.

Therefore, alcohol dependence is associated with variation in CHRNA5 mRNA levels while increased risk for nicotine dependence is associated with a missense variant in CHRNA5 that decreases response to a nicotine agonist. These results demonstrate that although variation in CHRNA5 influences risk for both alcohol dependence and nicotine dependence, different polymorphisms and different mechanisms of action are responsible for these effects on risk.

### Example 5

### CHRNA5 Expression Affects Risk for Nicotine Dependence

The correlation of two other SNPs, rs16969968 and rs514743, with nicotine dependence and CHRNA5 expression was assessed. The SNP haplotypes and their presence in nicotine addicted individuals and in control individuals are depicted in Table 4.

TABLE 4
Association of nicotine dependence with
haplotypes of rs16969968 and rs514743.

| Haplotypes | | | | | |
|---|---|---|---|---|---|
| rs16969968_{—} | Affected | Unaffected | | | |
| rs514743 | (Frequency) | (Frequency) | ChiSquare | DF | p value |
| GT | 0.37 | 0.39 | 0.31 | 1 | 0.5759 |
| AA | 0.40 | 0.29 | 10.75 | 1 | 0.0010 |
| GA | 0.23 | 0.32 | 8.50 | 1 | 0.0035 |

| | |
|---|---|
| AT | Haplotype does not exist |

The highest correlation between haplotypes and nicotine dependence was with haplotype AA where it is present in 40% of affected individuals, and 29% of controls. Comparing the GT and GA haplotypes where the haplotypes vary at the SNP rs514743 but not rs16969968, shows that the GA haplotype negatively correlates with nicotine dependence. The GA haplotype is present in 23% of nicotine addicted cases vs 32% in controls. Therefore, risk of nicotine dependence correletaes with different haplotypes of rs16969968 and rs514743, with the GA haplotype presenting the lowest risk (protective) of addiction, the GT haplotype being neutral, and the AA haplotype presenting the highest risk of nicotine addiction. The AT haplotype was not represented in the sampled individuals.

It was also found that expression of CHRNA5 was also affected in the three haplotypes, with the GA haplotype exhibiting low expression of the wild type allele, the GT haplotype exhibiting high expression of the wild type allele, and the AA haplotype exhibiting low expression of mutant allele.

### Example 6

### Additional CHRNA5-CHRNA3-CHRNB4 Gene Cluster Polymorphisms are Associated with Alcohol Dependence in Three Samples of Caucasians

Another screen was performed to identify additional polymorphisms in the CHRNA5-CHRNA3-CHRNB4 gene cluster that are associated with alcohol dependence. The data sets included the Collaborative Genetic Study of Nicotine Dependence (COGEND) data set, which was described in Bierut et al (2007), cocaine-dependent Caucasians and matched controls, and COGA independent case/control Caucasians, as described above.

All subjects were assessed and genotyped as described above in Example 1. Affected individuals were those who were alcohol dependent by DSM-IV criteria. The analysis included 1597 subject from COGEND, 256 cocaine- and alcohol-dependent subjects, and 963 COGA independent case/control Caucasian subjects. As shown in Table 5, two additional SNPs were identified that were associated with alcohol dependence. The data were adjusted for age and sex. SNP 40/rs8192475 and SNP 41/rs12914008 are nonsynonymous mutations (i.e., a codon is altered to code for another amino acid). SNP 40/rs8192475 is in exon 2 of the CHRNA3 gene and SNP 41/rs12914008 is in exon 4 the CHRNB4 gene. SNP 42/rs755203 is in the 5′ promoter region of the CHRNA4 gene.

TABLE 5
Association with Alcohol Dependence
in Three Samples of Caucasians.

| | | | | | | COGA | |
|---|---|---|---|---|---|---|---|
| | dbSNP | COGEND | | Cocaine* | | Case/Ctrl** | |
| SNP | reference | OR | p-value | OR | p-value | OR | p-value |
| 40 | rs8192475 | 0.54 | 0.047 | 0.41 | 0.058 | 0.59 | 0.044 |
| 41 | rs12914008 | 0.55 | 0.054 | 0.31 | 0.013 | 0.59 | 0.052 |
| 42 | rs755203 | 1.18 | 0.149 | 1.31 | 0.069 | 0.83 | 0.014 |

*Cocaine- and alcohol-dependent Caucasians.
**COGA independent case/control Caucasians.

### Example 7

### Polymorphisms Associated with CHRNA5 mRNA Expression

Our previous work using brain tissues derived from European subjects had suggested that risk for nicotine dependence and lung cancer is conferred by both mRNA expression levels and an amino acid change in CHRNA5. The risk allele of rs16969968 primarily occurs on the low mRNA expression allele of CHRNA5. The non-risk allele at rs16969968 occurs on both high and low expression alleles tagged by rs588765 within CHRNA5. When the non-risk allele occurs on the background of low mRNA expression of CHRNA5, the risk for nicotine dependence and lung cancer is significantly lower compared to those with the higher mRNA expression (Wang et al., 2009). There are 50 variants spanning 100 kb in the CHRNA5-A3-B4 gene cluster region that are highly correlated with rs588765 in European population. All of these variants are associated with a significant effect on mRNA expression levels in CHRNA5 (Table 6). However, it is not clear which of these variants are contributing directly to the changes in CHRNA5 mRNA expression. To narrow the number of putatively functional variants that affect the CHRNA5 mRNA expression, we performed quantitative allele specific expression. This method measures allele-specific transcript levels in the same individual; eliminating other biological variation that occurs when comparing expression levels between different samples. With this measurement we are able to determine whether the nicotine dependence associated variants have a cis-regulatory (direct) effect on CHRNA5 transcript levels and identify variant(s) that are responsible for modulating mRNA expression. We have completed our assays and our analysis has narrowed the region that harbors associated variants with mRNA expression from 100 kb to ˜25 kb upstream of CHRNA5, including the promoter region (FIG. 9).

It is known that populations of African descent have reduced linkage disequilibrium across this gene cluster. The contrasting genetic architecture in Africans and Europeans can be leveraged to identify the functional variant that alters CHRNA5 mRNA expression. We have recently performed quantitative total mRNA expression and allele specific expression assays in human frontal cortex tissues derived from African Americans' brain tissue. These assays confirm that CHRNA5 total RNA expression is strongly associated with variants located upstream of the gene in individuals of African ancestry as well as seen in individuals of European ancestry (Table 6). Furthermore, these assays provide strong evidence of possible functional variants in a region of ˜9 kb upstream of CHRNA5 alter mRNA levels of this gene (FIG. 10).

TABLE 6
Association of CHRNA5 mRNA expression with variants in ~25 kb upstream of the gene.

| | | | Individuals of | Individuals of | Individuals of |
|---|---|---|---|---|---|
| | | | European ancestry | African ancestry | African ancestry |
| SNP | position | gene | (Linear Refression) | (Linear Regression) | (Kruskal Walli test) |
| rs12916483 | 76619452 | upstream of PSMA4 | 2.83E−14 | 2.49E−03 | 1.59E−02 |
| | | transcription | | | |
| rs4886571 | 76620813 | intronic region of | 9.21E−14 | 3.14E−09 | 5.99E−06 |
| | | PSMA4 | | | |
| rs11858230 | 76622607 | upstream of CHRNA5 | 1.15E−13 | 1.71E−12 | 1.03E−06 |
| | | transcription | | | |
| rs8025429 | 76623417 | upstream of CHRNA5 | 1.07E−14 | 1.04E−12 | 1.03E−06 |
| | | transcription | | | |
| rs4887062 | 76624856 | upstream of CHRNA5 | 4.65E−14 | 1.87E−13 | 2.27E−07 |
| | | transcription | | | |
| rs8053 | 76628275 | upstream of CHRNA5 | 1.17E−13 | 1.03E−15 | 2.12E−08 |
| | | transcription | | | |
| rs1979907 | 76629294 | upstream of CHRNA5 | 4.65E−14 | 1.03E−15 | 2.12E−08 |
| | | transcription | | | |
| rs1979906 | 76629344 | upstream of CHRNA5 | 4.65E−14 | 8.10E−15 | 1.36E−07 |
| | | transcription | | | |
| rs1979905 | 76629429 | upstream of CHRNA5 | 4.65E−14 | 1.03E−15 | 2.12E−08 |
| | | transcription | | | |
| rs12907966 | 76630106 | upstream of CHRNA5 | 1.59E−14 | not | not |
| | | transcription | | polymorphic | polymorphic |
| rs880395 | 76631411 | upstream of CHRNA5 | 4.65E−14 | 1.03E−15 | 2.12E−08 |
| | | transcription | | | |
| rs905740 | 76631441 | upstream of CHRNA5 | 4.65E−14 | 2.56E−15 | 1.67E−08 |
| | | transcription | | | |
| rs7164030 | 76631716 | upstream of CHRNA5 | 8.34E−14 | 8.41E−17 | 5.40E−08 |
| | | transcription | | | |
| rs4275821 | 76636596 | upstream of CHRNA5 | 1.19E−08 | 6.05E−11 | 8.21E−07 |
| | | transcription | | | |
| rs3841324 | 76644868 | promoter of CHRNA5 | 1.19E−14 | 1.31E−02 | 1.92E−02 |
| rs588765 | 76652480 | intronic region of | 1.39E−11 | 1.64E−08 | 1.31E−05 |
| | | CHRNA5 | | | |
| rs601079 | 76656634 | intronic region of | 1.24E−11 | 8.90E−05 | 3.99E−03 |
| | | CHRNA5 | | | |
| rs615470 | 76673043 | 3′UTR of CHRNA5 | 4.91E−06 | 7.80E−06 | 1.30E−04 |

What is claimed is:

1. A method for identifying a subject at risk for substance dependence, the method comprising detecting in a sample from the subject the presence of at least one polymorphism in the CHRNA5-CHRNA3-CHRNB4 gene cluster and the CHRNA4 gene, the polymorphisms having a correlation value of 0.7 or greater with each other, wherein the presence of one of the alleles of the polymorphism is associated with increased risk for substance dependence.

2. The method of claim 1, wherein the at least one polymorphism is selected from the group consisting SNP 1, SNP 2, SNP 3, SNP 6, SNP 11, SNP 12, SNP 15, SNP 17, SNP 19, SNP 23, SNP 30, SNP 31, SNP 32. SNP 40, SNP, 41, SNP 42, SNP 43, SNP 44, SNP 45, SNP46 and a combination thereof.

3. The method of claim 1, wherein the at least one polymorphism is selected from the group consisting of:
SNP 6, wherein the absence of a 22 base pair insertion is associated with increased risk for substance dependence;
SNP 11, wherein the presence of A rather than T is associated with increased risk for substance dependence;
SNP 12, wherein the presence of A rather than G is associated with increased risk for substance dependence;
SNP 15, wherein the presence of G rather than C is associated with increased risk for substance dependence;
SNP 19, wherein the presence of T rather than C is associated with increased risk for substance dependence;
SNP 40, wherein the presence of G rather than A is associated with increased risk for substance dependence;
SNP 41, wherein the presence of G rather than A is associated with increased risk for substance dependence;
SNP 42, wherein the presence of T is associated with increased risk for substance dependence;
SNP 43, wherein the presence of A is associated with increased risk for substance dependence;
SNP 44, wherein the presence of A is associated with increased risk for substance dependence;
SNP 45, wherein the presence of T is associated with increased risk for substance dependence;
SNP 46, wherein the presence of T is associated with increased risk for substance dependence; and
a combination thereof.

4. The method of claim 1, wherein the polymorphism is SNP 6.

5. The method of claim 1, wherein the polymorphism is detected with at least one oligonucleotide that distinguishes between two alternate alleles of the polymorphism.

6. The method of claim 5, wherein the oligonucleotide is used in method selected from the group consisting of an amplification method, a hybridization method, a sequencing method, and a combination thereof.

7. The method of claim 1, wherein the substance dependence is selected from the group consisting of alcohol dependence, cocaine dependence, and heroin dependence.

8. The method of claim 1, wherein the substance dependence is alcohol dependence.

9. A method for determining the response of a subject to a therapeutic substance, the method comprising detecting in a sample from the subject the presence of at least one polymorphism in the CHRNA5-CHRNA3-CHRNB4 gene cluster and the CHRNA4 gene, wherein the presence of a first allele of the polymorphism is associated with a first response and the presence of a second allele of the polymorphism is associated with a second response.

10. The method of claim 9, wherein the polymorphism has correlation value of 0.7 or greater with the polymorphisms in the CHRNA5-CHRNA3-CHRNB4 gene cluster and the CHRNA4 gene.

11. The method of claim 9, wherein the polymorphism is selected from the group consisting SNP 1, SNP 2, SNP 3, SNP 6, SNP 11, SNP 12, SNP 15, SNP 17, SNP 19, SNP 23, SNP 30, SNP 31, SNP 32. SNP 40, SNP, 41, SNP 42, SNP 43, SNP 44, SNP 45, SNP 46 and a combination thereof.

12. The method of claim 9, wherein the first or second response is selected from the group consisting of sensitivity to the therapeutic substance and adverse reactions due to the therapeutic substance.

13. The method of claim 9, wherein the therapeutic substance is selected from the group consisting of a nicotinic acetylcholine receptor agonist, a nicotinic acetylcholine receptor partial agonist, and a nicotinic acetylcholine receptor antagonist.

14. The method of claim 13, wherein the nicotinic acetylcholine receptor agonist is selected from the group consisting of carbamylcholine, methylcarbamylcholine, epibatidine, epiboxidine, and altinicline.

15. The method of claim 13, wherein the nicotinic acetylcholine receptor partial agonist is selected from the group consisting of varenicline, isopronidine, tropisetron, cytsine, and imidacloprid.

16. The method of claim 13, wherein the nicotinic acetylcholine receptor antagonist is selected from the group consisting of bupropion, hexamethonium, mecamylamine, fluoxetine, and iptakalim.

17. A method for evaluating the response of a subject to a substance cessation treatment, the method comprising determining the level of CHRNA5 messenger RNA in a first and a second sample from the subject, the first sample collected before the start of the substance cessation treatment, the second sample collected during or after the substance cessation treatment, wherein a decreased level of CHRNA5 messenger RNA in the second sample relative to the first sample indicates the subject is responding to the substance cessation treatment.

18. A kit for genotyping a subject for at least one polymorphism in the CHRNA5-CHRNA3-CHRNB4 gene cluster and the CHRNA4 gene, the kit comprising at least one oligonucleotide that distinguishes between two alleles of the at least one polymorphism.

19. The kit of claim 18, wherein the at least one oligonucleotide is selected from the group consisting of:
at least one oligonucleotide that distinguishes between the long allele and the short allele of SNP 6;
at least one oligonucleotide that distinguishes between the A allele and the T allele of SNP 11;
at least one oligonucleotide that distinguishes between the A allele and the G allele of SNP 12;
at least one oligonucleotide that distinguishes between the C allele and the G allele of SNP 15;
at least one oligonucleotide that distinguishes between the C allele and the T allele of SNP 19;
at least one oligonucleotide that distinguishes between the G allele and the A allele of SNP 40;
at least one oligonucleotide that distinguishes between the G allele and the A allele of SNP 41;
at least one oligonucleotide that recognizes the T allele of SNP 42; and
a combination thereof.
